SEQUENCE LISTING (2 sequences)

SEQ ID NO: 1 - protein, 13 aa, synthetic construct
  NON_STD 2: note = N-Methylaniline
  NON_STD 7: note = Hydroxyproline
  NON_STD 11..12: note = N-methyl Norleucine
YXNPHLXWXXKCG

SEQ ID NO: 2 - protein, 15 aa, synthetic construct
  NON_STD 2: note = Methylaniline
  NON_STD 7: note = Hydroxyproline
  NON_STD 11..12: note = N-methyl Norleucine
YXNPHEXWSWXXKCG

(19) United States
(12) Patent Application Publication
(10) Pub. No.: US 2025/0135047 A1
(43) Pub. Date: May 1, 2025

(54) IMPROVED ALUMINUM FLUORIDE RADIOSYNTHESIS OF [18F]DK222

(71) Applicant: The Johns Hopkins University, Baltimore, MD (US)

(72) Inventors: Sridhar NIMMAGADDA, Baltimore, MD (US); Daniel P. HOLT, Severna Park, MD (US); Robert F. DANNALS, Sparks, MD (US); Martin Gilbert POMPER, Baltimore, MD (US); Dhiraj KUMAR, Baltimore, MD (US)

(21) Appl. No.: 18/681,241

(22) PCT Filed: Aug. 5, 2022
(86) PCT No.: PCT/US2022/039621

§ 371 Date: Feb. 5, 2024

Related U.S. Application Data
(60) Provisional application No. 63/230,338, filed on Aug. 6, 2021.

(51) Int. Cl. A61K 51/08; A61K 51/04

(52) U.S. Cl. CPC A61K 51/088; A61K 51/0482; A61K 2123/00

(57) ABSTRACT

A method for preparing an ¹⁸F-labeled imaging agent comprising a conjugate of a peptide having a binding specificity for programmed death ligand 1 (PD-L1) and a chelating moiety, and optionally a linker. In representative aspects, the ¹⁸F-labeled imaging agent is [¹⁸F]-DK222.

### FEDERALLY SPONSORED RESEARCH OR DEVELOPMENT

This invention was made with government support under grant CA236616 and EB024495 awarded by the National Institutes of Health. The government has certain rights in the invention.

### SEQUENCE LISTING

The text of the computer readable sequence listing filed herewith, titled “39714-601_SEQUENCE_LISTING”, created Aug. 5, 2022, having a file size of 5,010 bytes, is hereby incorporated by reference in its entirety.

### BACKGROUND

Programmed death ligand-1 (PD-L1) is a protein that is highly expressed by several cancers as an immune evasion mechanism. PD-L1 levels in the tumor microenvironment detected by immunohistochemistry (IHC) is an FDA-approved companion diagnostic test to guide therapeutics targeting PD-L1 and its receptor PD-1. Davis and Patel, 2019. Although effective, PD-L1 IHC falls short in reporting on the inter-and intra-tumor heterogeneity in PD-L1 expression, which hampers the ability to personalize immunotherapies to improve outcomes. Mclaughlin et al., 2016; Gandini et al., 2016.

To address this unmet need, a small peptide-based imaging agent for PD-L1 that is radiolabeled with fluorine-18, referred to herein as [¹⁸F]DK222, for positron emission tomography imaging (PET) was developed. See, e.g., Kumar et al., 2019. [¹⁸F]DK222 recently moved to evaluation in patients by positron emission tomography (PET). Johns Hopkins, 2021. Thus, the efficient production of [¹⁸F]DK222 using large amounts of radioactivity is paramount for its wide distribution and eventual use in patients across a spectrum of cancers.

### SUMMARY

In some aspects, the presently disclosed subject matter provides a method for preparing an ¹⁸F-labeled imaging agent comprising a conjugate of a peptide having a binding specificity for programmed death ligand 1 (PD-L1) and a chelating moiety, and optionally a linker, wherein the linker, when present connects the peptide and the chelating moiety, and when the linker is absent, the chelating moiety is attached directly to the peptide through a primary amine of an amino acid of the peptide, the method comprising: (a) providing or preparing a peptide conjugate precursor solution comprising a conjugate of a peptide having a binding specificity for programmed death ligand 1 (PD-L1) and a chelating moiety, and optionally a linker; (b) providing or preparing an anion exchange cartridge comprising [¹⁸F] ions; (c) adding an aluminum chloride solution, acetonitrile, ascorbic acid, and the peptide precursor solution to a reaction vessel; (d) eluting the anion exchange cartridge to release the [¹⁸F] ions into the reaction vessel; (e) heating the reaction vessel for a period of time; (f) cooling the reaction vessel; (g) adding ascorbic acid to the cooled reaction vessel to form a solution comprising the ¹⁸F-labeled imaging agent; (h) eluting the solution comprising the ¹⁸F-labeled imaging agent through a solid phase extraction cartridge; (i) washing the solid phase extraction cartridge with ascorbic acid; (j) eluting the solid phase extraction cartridge with an alkyl alcohol followed by sodium chloride for injection, USP with (+)-sodium L-ascorbate, through a sterilizing filter; and (k) collecting the ¹⁸F-labeled imaging agent.

In some aspects, the peptide having a binding specificity for PD-L1 interacts with amino acids Y56, E58, A113, M115, and Y123 of PD-L1. In certain aspects, the peptide having a binding specificity for programmed death ligand 1 (PD-L1) has at least 80% sequence identity to the peptide WL12, DK221, or DK222. In certain aspects, the peptide having a binding specificity for programmed death ligand 1 (PD-L1) has at least 85% sequence identity to the peptide WL12, DK221, or DK222. In certain aspects, the peptide having a binding specificity for programmed death ligand 1 (PD-L1) imaging agent has at least 90% sequence identity to the peptide WL12, DK221, or DK222. In certain aspects, the peptide having a binding specificity for programmed death ligand 1 (PD-L1) has 100% sequence identity to the peptide WL12, DK221, or DK222.

In particular aspects, the imaging agent comprises a compound of formula (I):

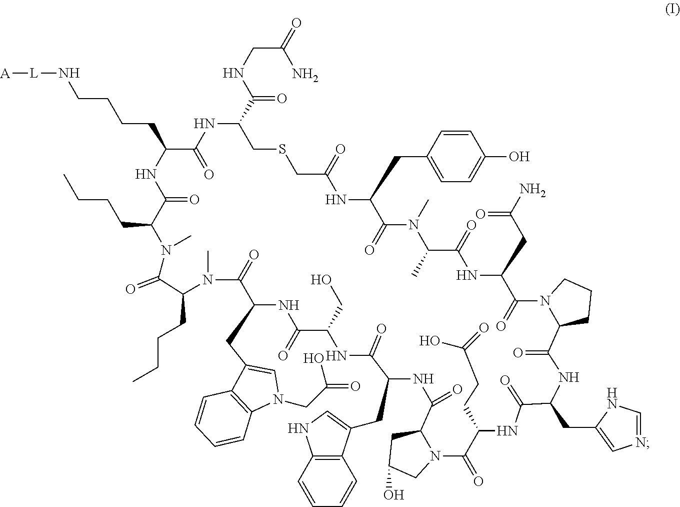

wherein:
L is a linker, which can be present or absent, and when present has the following general formula:

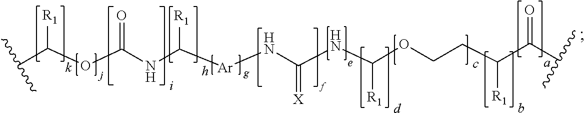

wherein:
X is S or O;
a, e, f, g, i, and j are each independently an integer selected the group consisting of 0 and 1;
b, d, h, and k are each independently an integer selected from the group consisting of 0, 1, 2, 3, 4, 5, 6, 7, and 8;
c is an integer having a range from 0 to 40;
each R₁ is H or —COOR₂, wherein R₂ is H or C₁-C₄ alkyl; Ar is substituted or unsubstituted aryl or heteroaryl; and
A is a chelating moiety.

In certain aspects, the linker is selected from the group consisting of:

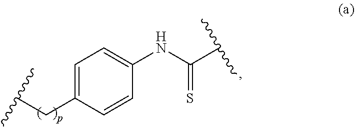

wherein p is an integer selected from 0, 1, 2, 3, and 4;

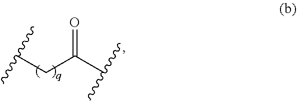

wherein q is an integer selected from the group consisting of 0, 1, 2, 3, 4, 5, 6, 7, and 8;

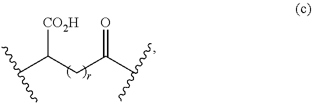

wherein r is an integer selected from the group consisting of 0, 1, 2, 3, 4, 5, 6, 7, and 8;

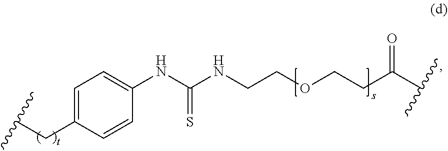

wherein s is an integer having a range from 1 to 40 and t is an integer selected from 0 or 1;

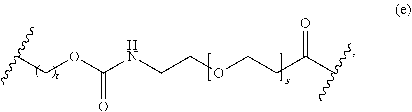

wherein s is an integer having a range from 1 to 40 and t is an integer selected from 0 or 1; and

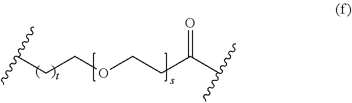

wherein s is an integer having a range from 1 to 40 and t is an integer selected from 0 or 1.

In particular aspects, the chelating moiety is selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTA-tris(t-butyl)ester, DOTAGA-(t-butyl)₄, DOTA-di(t-butyl)ester, DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), DO3A-(t-butyl), DO3AM (2,2′,2″-(1,4,7,10-tetraazacyclododecane-1,4,7-triyl)triacetamide), p-NH₂-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NOTA(t-butyl)₂, NO2A (1,4,7-Triazacyclononane-1,4-bis(acetic acid)-7-(acetamide), NODA (1,4,7-triazacyclononane-1,4-diacetate); NODAGA (1,4,7-triazacyclononane, 1-glutaric acid-4,7-acetic acid), NODAGA(t-butyl)₃, NOTAGA (1,4,7-triazonane-1,4-diyl)diacetic acid), DFO (Desferoxamine), DTPA (2-[Bis[2-[bis(carboxymethyl)amino]ethyl]amino]acetic acid), DTPA-tetra(t-butyl)ester (diethylenetriamine-N,N,N″,N″-tetra-tert-butyl acetate-N′-acetic acid), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3,6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), BaBaSar, tris(hydroxypyridinone) (THP), THP(benzyl)₃, NOPO (3-(((4,7-bis((hydroxy(hydroxymethyl)phosphoryl)-methyl)-1,4,7-triazonan-1-yl)methyl)(hydroxy)phosphoryl)propanoic acid), TRAP (3,3′,3″-(((1,4,7-triazonane-1,4,7-triyl)tris(methylene))tris(hydroxyphosphoryl))-tripropanoic acid), p-NH₂-Bn-PCTA (3,6,9,15-Tetraazabicyclo[9.3.1] pentadeca-1(15),11,13-triene-4-S-(4-aminobenzyl)-3,6,9-triacetic acid), and biotin (5-[(3aS,4S,6aR)-2-oxohexahydro-1H-thieno[3,4-d]imidazol-4-yl]pentanoic acid).

In more particular aspects, the chelating moiety is selected from the group consisting of:

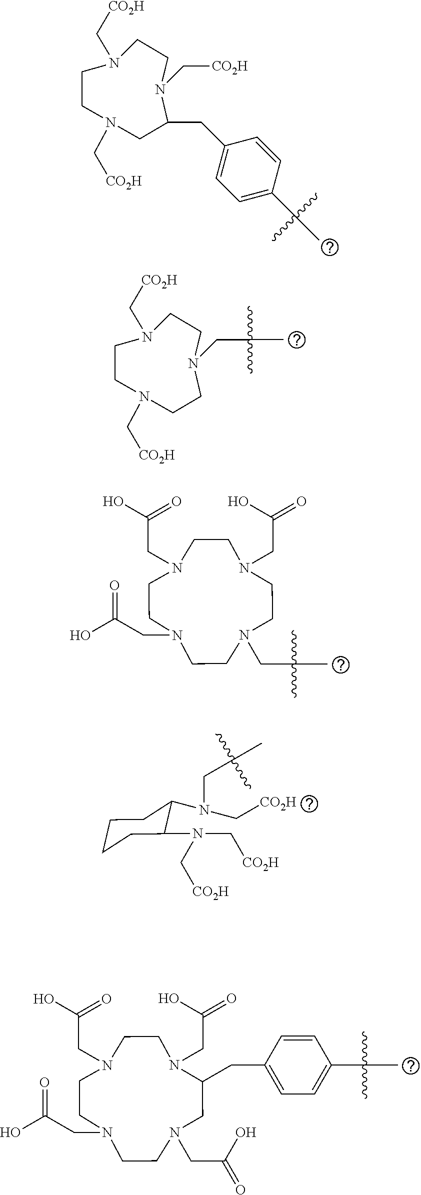 [SMILES: *c1ccc(CC2CN(CC(=O)O)CCN(CC(=O)O)CCN2CC(=O)O)cc1 ; *CN1CCN(CC(=O)O)CCN(CC(=O)O)CC1 ; *CN1CCN(CC(=O)O)CCN(CC(=O)O)CCN(CC(=O)O)CC1 ; *CN(CC(=O)O)C1CCCCC1N(CC(=O)O)CC(=O)O ; *c1ccc(CC2CN(CC(=O)O)CCN(CC(=O)O)CCN(CC(=O)O)CCN2CC(=O)O)cc1]

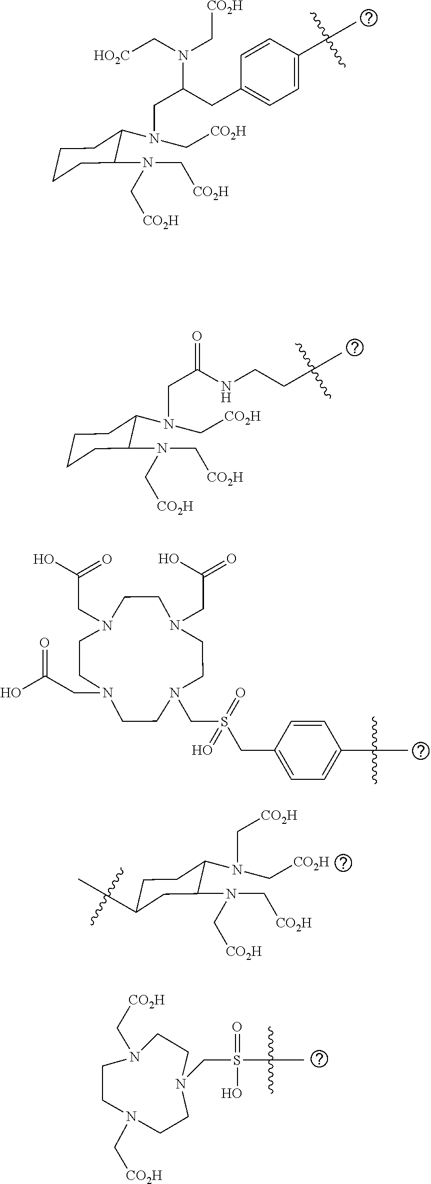

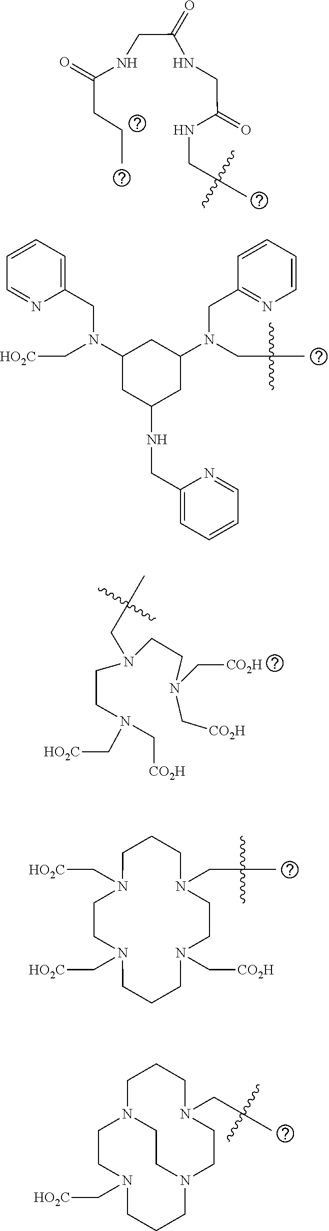 [SMILES: *CNC(=O)CNC(=O)CNC(=O)CCC ; *CN(Cc1ccccn1)C1CC(NCc2ccccn2)CC(N(CC(=O)O)Cc2ccccn2)C1 ; *CN(CCN(CC(=O)O)CC(=O)O)CCN(CC(=O)O)CC(=O)O ; *CN1CCCN(CC(=O)O)CCN(CC(=O)O)CCCN(CC(=O)O)CC1 ; *CN1CCCN2CCN(CCCN(CC(=O)O)CC2)CC1]

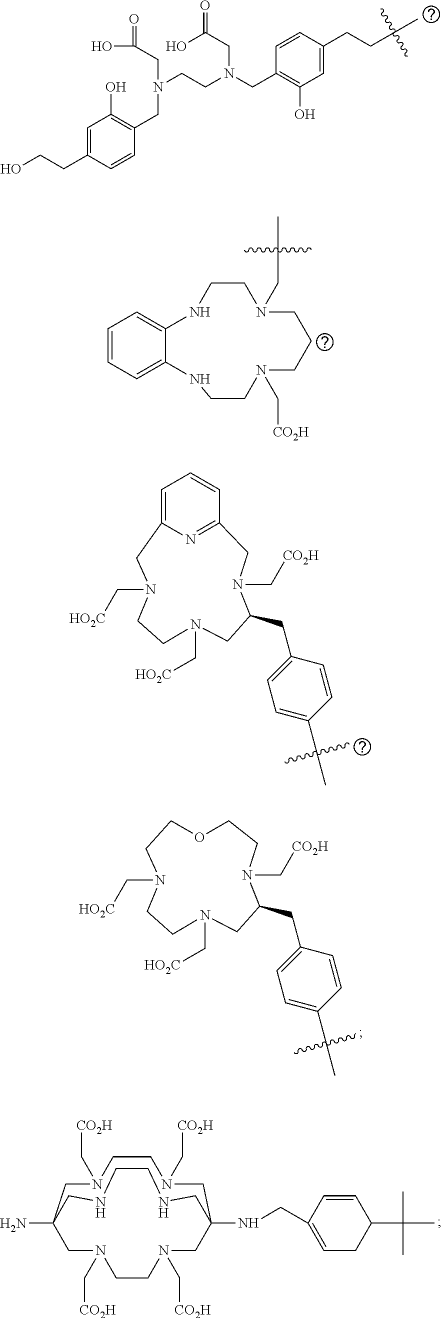 [SMILES: *CCc1ccc(CN(CCN(CC(=O)O)Cc2ccc(CCO)cc2O)CC(=O)O)c(O)c1 ; *CN1CCCN(CC(=O)O)CCNc2ccccc2NCC1 ; *c1ccc(C[C@H]2CN(CC(=O)O)CCN(CC(=O)O)Cc3cccc(n3)CN2CC(=O)O)cc1 ; *c1ccc(C[C@H]2CN(CC(=O)O)CCN(CC(=O)O)CCOCCN2CC(=O)O)cc1 ; CC(C)(C)C1C=CC(CNC23CNCCNCC(N)(CN(CC(=O)O)CCN(CC(=O)O)C2)CN(CC(=O)O)CCN(CC(=O)O)C3)=CC1]

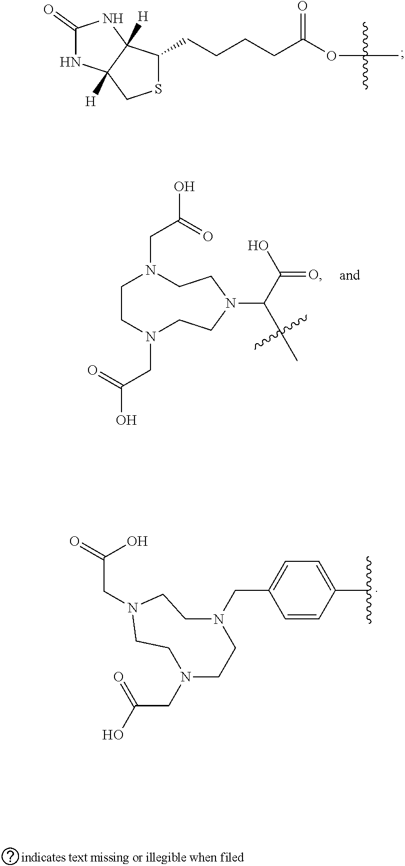 [SMILES: *OC(=O)CCCC[C@@H]1SC[C@@H]2NC(=O)N[C@@H]21 ; *C(C(=O)O)N1CCN(CC(=O)O)CCN(CC(=O)O)CC1 ; *c1ccc(CN2CCN(CC(=O)O)CCN(CC(=O)O)CC2)cc1]

In yet more particular aspects, the chelating moiety is selected from the group consisting of DOTA (1,4,7,10-tetraazacyclododecane-1,4,7,10-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane, 1-glutaric acid-4,7-acetic acid), and biotin (5-[(3aS,4S,6aR)-2-oxohexahydro-1H-thieno[3,4-d]imidazol-4-yl]pentanoic acid).

In certain aspects, the imaging agent is selected from the group consisting of:

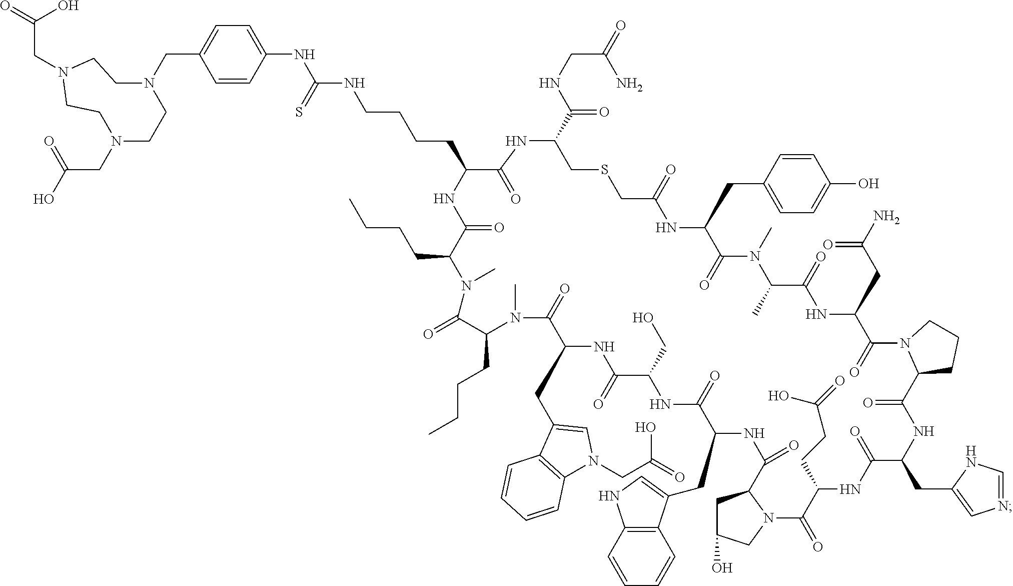 [SMILES: CCCC[C@H]1C(=O)N(C)[C@@H](CCCC)C(=O)N[C@@H](CCCCNC(=S)Nc2ccc(CN3CCN(CC(=O)O)CCN(CC(=O)O)CC3)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

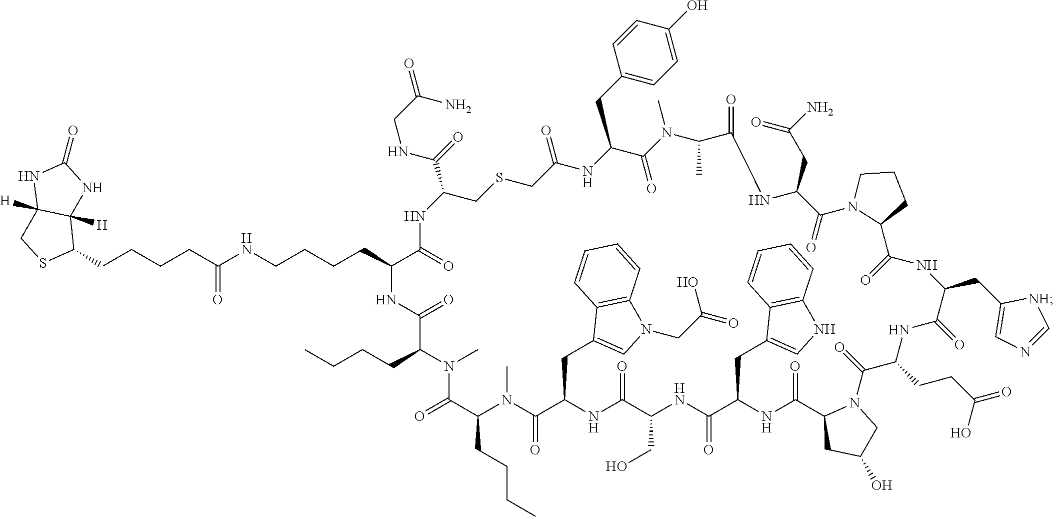 [SMILES: CCCC[C@H]1C(=O)N(C)[C@@H](CCCC)C(=O)N[C@@H](CCCCNC(=O)CCCC[C@@H]2SC[C@@H]3NC(=O)N[C@@H]32)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@H](CCC(=O)O)C(=O)N2C[C@H](O)C[C@H]2C(=O)N[C@H](Cc2c[nH]c3ccccc23)C(=O)N[C@H](CO)C(=O)N[C@H](Cc2cn(CC(=O)O)c3ccccc23)C(=O)N1C]

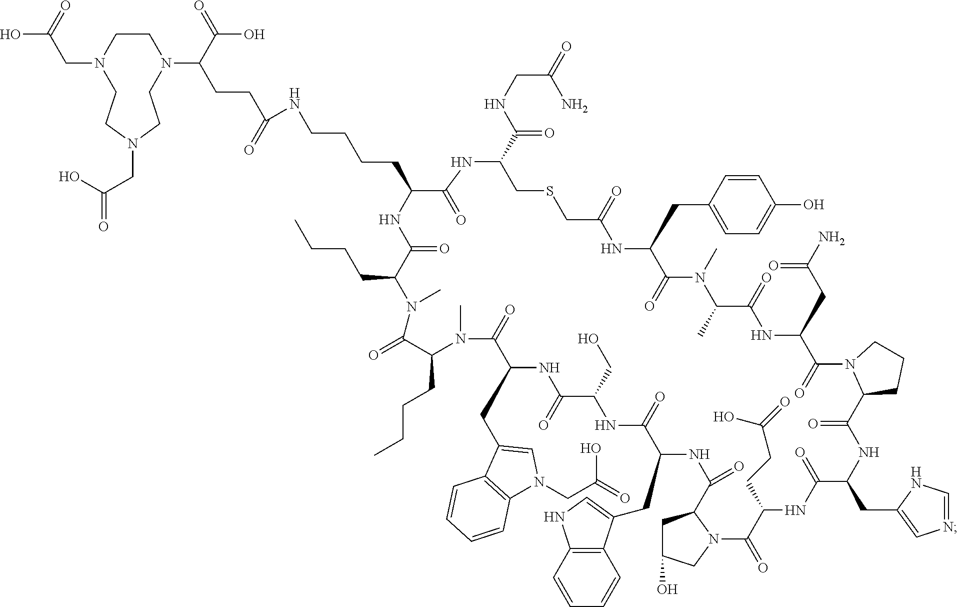 [SMILES: CCCC[C@H]1C(=O)N(C)[C@@H](CCCC)C(=O)N[C@@H](CCCCNC(=O)CCC(C(=O)O)N2CCN(CC(=O)O)CCN(CC(=O)O)CC2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

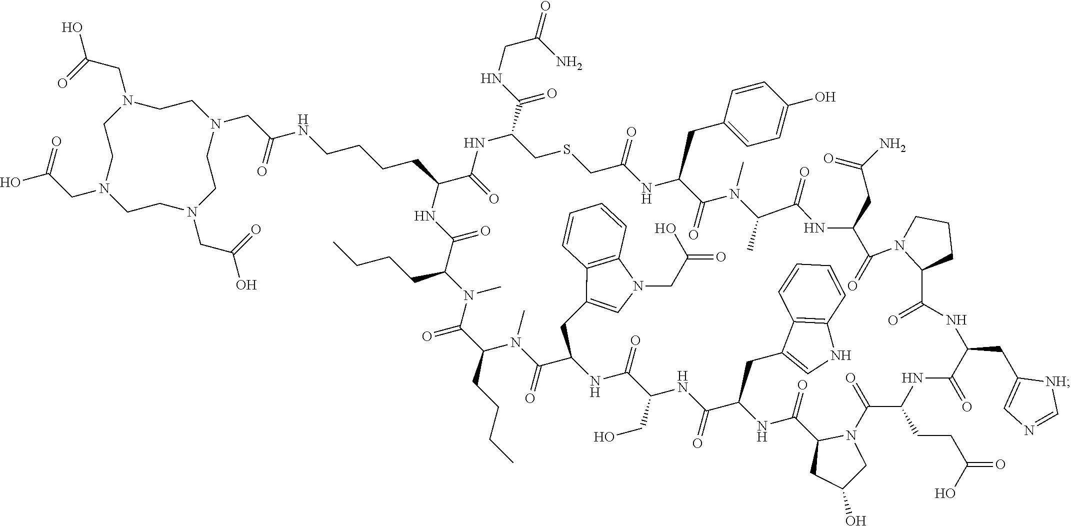 [SMILES: CCCC[C@H]1C(=O)N(C)[C@@H](CCCC)C(=O)N[C@@H](CCCCNC(=O)CN2CCN(CC(=O)O)CCN(CC(=O)O)CCN(CC(=O)O)CC2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@H](CCC(=O)O)C(=O)N2C[C@H](O)C[C@H]2C(=O)N[C@H](Cc2c[nH]c3ccccc23)C(=O)N[C@H](CO)C(=O)N[C@H](Cc2cn(CC(=O)O)c3ccccc23)C(=O)N1C]

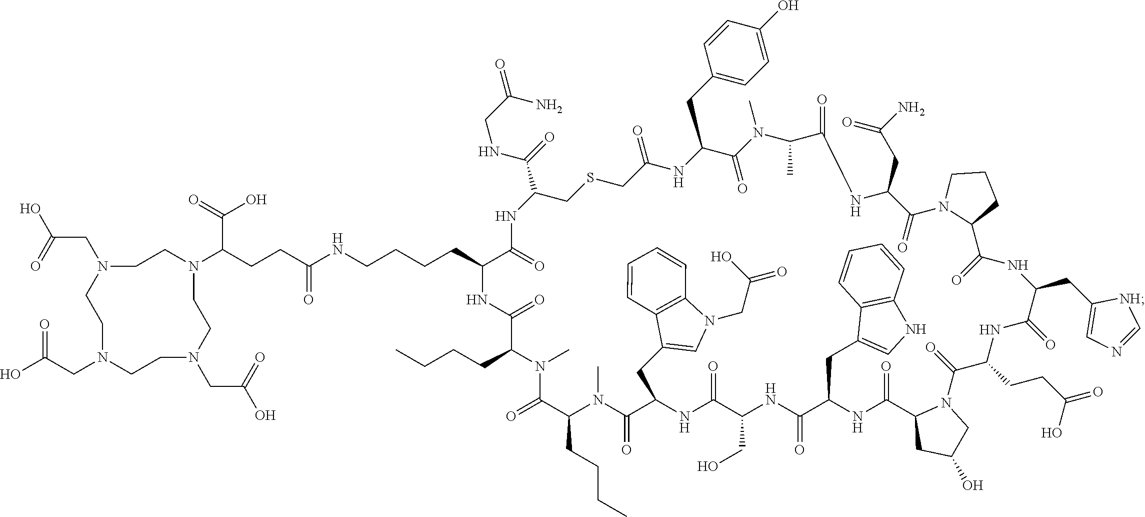

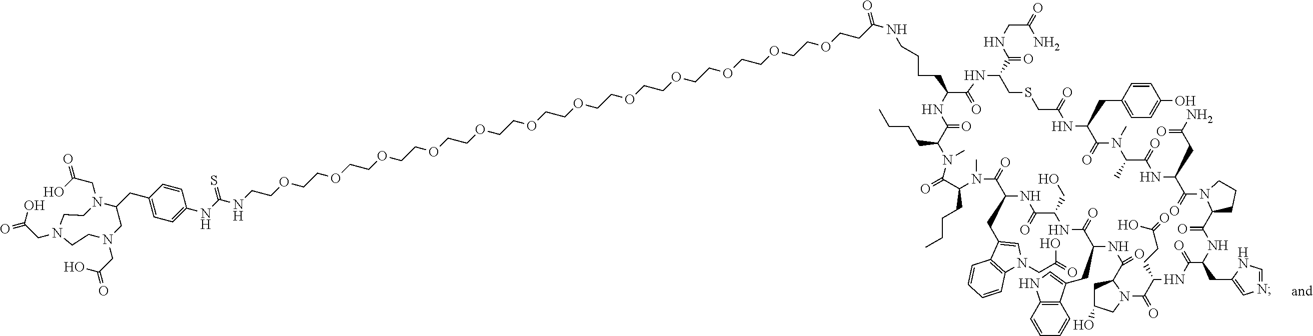 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCNC(=S)Nc2ccc(CC3CN(CC(=O)O)CCN(CC(=O)O)CCN3CC(=O)O)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

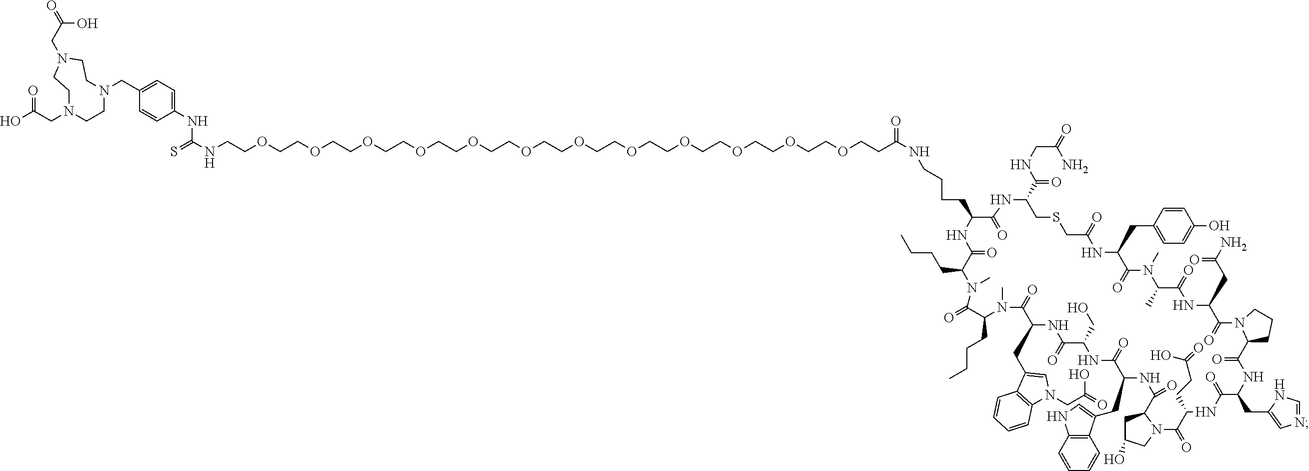 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCNC(=S)Nc2ccc(CN3CCN(CC(=O)O)CCN(CC(=O)O)CC3)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

wherein the chelating moiety of each compound comprises Al[¹⁸F].

In more certain aspects, the ¹⁸F-labeled imaging agent is [¹⁸F]DK222 having the following chemical structure:

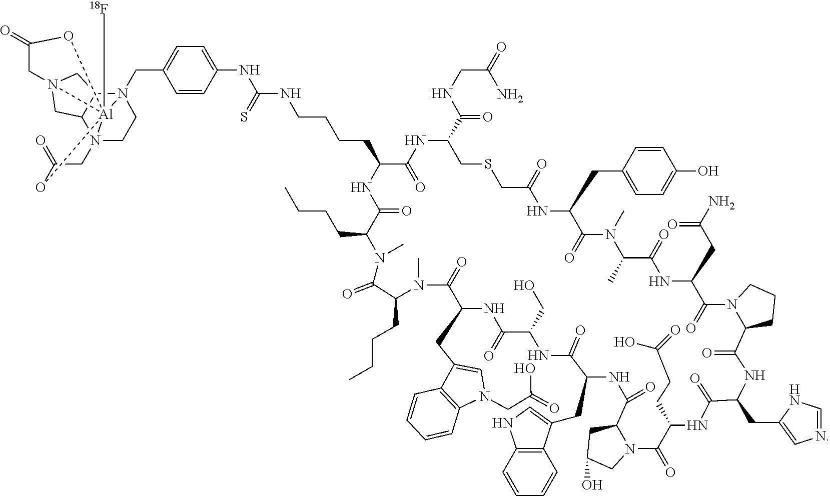

In some aspects, the aluminum chloride solution comprises an aluminum chloride hexahydrate solution in ascorbic acid. In some aspects, the ascorbic acid at each occurrence comprises a 5-M ascorbic acid aqueous solution. In some aspects, the anion exchange cartridge is eluted with a base. In some aspects, the base is potassium acetate. In some aspects, the reaction vessel is heated to about 110° C.±20° C.

In certain aspects, the reaction vessel is heated for about 10 minutes. In certain aspects, the reactive vessel is cooled to less than about 50° C.

In some aspects, the solid phase extraction cartridge comprises a C18 or tC18 sorbent.

In some aspects, the alkyl alcohol is selected from the group consisting of methanol, ethanol, propyl alcohol, isopropyl alcohol, and butanol. In particular aspects, the alkyl alcohol is ethanol.

In some aspects, the sterilizing filter comprises a polytetrafluoroethylene membrane.

In particular aspects, one or more of steps (a)-(k) are automated.

In some aspects, the ¹⁸F-labeled imaging agent meets one or more quality control criteria selected from the group consisting of: (a) a clear, colorless solution with no visible particulate matter; (b) a yield not less than 20 mCi of the ¹⁸F-labeled imaging agent; (c) a pH in the range of 3.5 to 5.5; (d) the sterilizing filter has a greater than or equal to 13 psi bubble-point test; (e) a radionuclidic identity and purity in the range of 105-115 minutes for half-life measurement; (f) a radiochemical purity of 90% or greater for the ¹⁸F-labeled imaging agent; (g) a specific activity greater than 250 mCi/μmole; (h) less than 10% ethanol and less than 273 parts per million acetonitrile residual solvent; and (i) bacterial endotoxins equal to or less than eleven USP endotoxin units per mL.

In certain aspects, the method further comprises inoculating the ¹⁸F-labeled imaging agent at the end of each production day and monitoring for 14 days for visible growth of microorganisms.

In some aspects, the method comprises a starting radioactivity of the [¹⁸F] ions comprising the anion exchange cartridge having a range from about 100 mCi to greater than about 1,000 mCi. In some aspects, the ¹⁸F-labeled imaging agent has a radioactivity having a range from about 50 mCi to greater than about 500 mCi. In some aspects, the ¹⁸F-labeled imaging agent has a yield at the end of synthesis (EOS) having a range from about 30% to about 60%. In some aspects, the ¹⁸F-labeled imaging agent has a specific activity having a range from about 2,000 mCi/μmole to greater than about 25,000 mCi/μmole. In some aspects, the ¹⁸F-labeled imaging agent has an effective specific activity having a range from about 900 mCi/μmole to about 8,000 mCi/μmole. In some aspects, the ¹⁸F-labeled imaging agent has a radiochemical activity having a range from about 90% to about 100%.

In some aspects, the presently disclosed subject matter provides a kit for detecting Programmed Death Ligand 1 (PD-L1), the kit comprising a peptide conjugate precursor solution comprising a conjugate of a peptide having a binding specificity for programmed death ligand 1 (PD-L1) and a chelating moiety, and optionally a linker.

In certain aspects, the kit further comprises an anion exchange cartridge comprising [¹⁸F] ions. In certain aspects, the kit further comprises a solid phase extraction cartridge. In certain aspects, the kit further comprises one or more solvents or reagents necessary for preparing an ¹⁸F-labeled imaging agent by the presently disclosed methods.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 shows structures of [¹⁸F]DK222 and its parent peptide DK222;

FIG. 2 shows an apparatus for the automated purification and reformulation of [¹⁸F]DK222;

FIG. 3 is schematic diagram of the automated synthesis of DK222 using a FASTlab 2 PET synthesis module;

FIG. 4 is a representative HPLC chromatogram demonstrating the quality control of sample #9;

FIG. 5 is a representative HPLC chromatogram demonstrating the stability of sample #9 over a 4-hour period;

FIG. 6A is a plot of the non-decay corrected (ndc) yield versus starting activity (mCi) for the synthesis of DK222 with a custom-built SPE module; and

FIG. 6B is a plot of the non-decay corrected (ndc) yield versus starting activity (mCi) for the synthesis of DK222 with a commercial FASTlab 2 module.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Improved Aluminum Fluoride Radiosynthesis of [¹⁸F]DK222

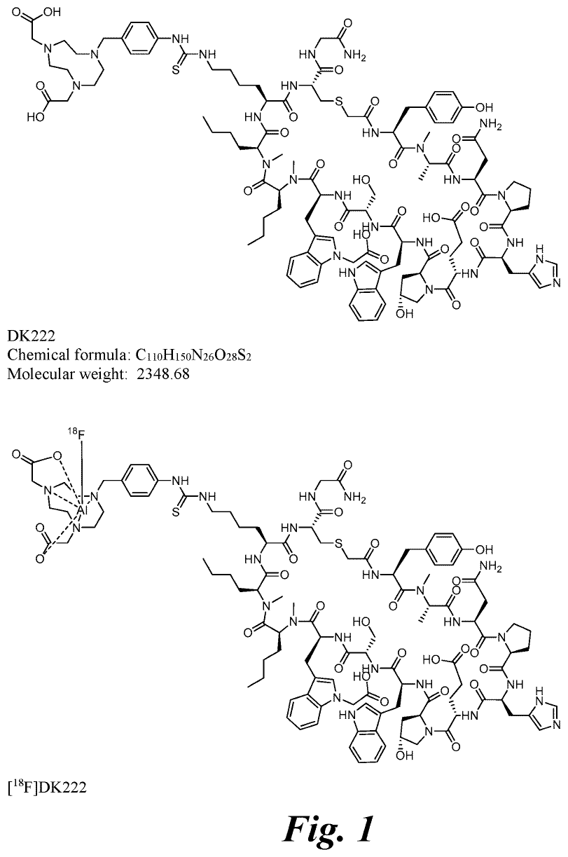

The presently disclosed subject matter provides the development, optimization, and validation of an automated synthesis procedure for [¹⁸F]DK222 that would facilitate production at a central facility and its distribution and use in patients. Referring now to FIG. 1, the structures of [¹⁸F]DK222 and its parent peptide DK222 are shown. See also U.S. Patent Application Publication No. 2019/0314531, for TUMOR AND IMMUNE CELL IMAGING BASED ON PD-L1 EXPRESSION, to Nimmagadda et al., published Oct. 17, 2019; and International PCT Patent Application Publication No. WO/2022/032100 for IMAGING AND TARGETING PROGRAMMED DEATH LIGAND-1 (PD-L1) EXPRESSION, to Nimmagadda et al., published Feb. 10, 2022, each of which is incorporated herein by reference in their entirety.

[¹⁸F] is a conspicuous radionuclide in the clinic. The 110-min half-life of [¹⁸F] facilitates synthesis of radiopharmaceuticals at a central radiopharmacy for distribution to regional and local imaging centers for PET imaging. Jacobson et al., 2015. Central radiopharmacy production of imaging agents comprising [¹⁸F] requires starting a synthesis with large amounts of radioactivity that is often in the hundreds of millicurie or more than a Curie. Large scale radiopharmaceutical production also demands robust and reproducible synthesis methods, as well as long-term stability (4 h or more) of the produced radiopharmaceutical at higher radioactivity exposures.

[¹⁸F]DK222 has been previously synthesized by the aluminum [¹⁸F]fluoride (Al[¹⁸F]) method known in the art. McBride et al., 2010. The Al[¹⁸F] method has been increasingly used for radiopharmaceutical preparation of peptides and small molecules. McBride et al., 2010; Wan et al., 2013; D'Souza et al., 2011; Lutje et al., 2019; Kumar and Ghosh, 2018. As a result, Al[¹⁸F]-labelled radiopharmaceuticals have been used in several first-in-human and early phase clinical trials. Wan et al., 2013; Liu et al., 2019; Yu et al., 2015.

The Al[¹⁸F] methods known in the art for the large scale preparation and distribution of radiopharmaceuticals are hampered, however, by poor radiochemical yields and satiability of the synthesized radiopharmaceuticals when a high amount of [¹⁸F] is used. While a few automation methods and kit formulations have been reported, they have not overcome the abovementioned limitations, therefore making them unsuitable for the large scale production of peptide-based imaging agents. Wan et al., 2013; Tshibangu et al., 2020; Giglio et al., 2018. Some advances have been made in the area of small molecule radiotracers, but at the cost of low radiochemical yields. Kersemans et al., 2018. Al[¹⁸F] methods for the successful large-scale production of peptide-based imaging agents, however, have not been reported.

Accordingly, in some embodiments, the presently disclosed subject matter provides methods for the improved Al[¹⁸F] synthesis of the peptide-based imaging agent, [¹⁸F]DK222, including methods of quality control for the synthesis and final product. The previous radiosynthesis of [¹⁸F]DK222 was a manual method, which required hands-on manipulation for the synthesis, as well as for the purification and final formulation. The presently disclosed synthesis method has been optimized to reduce radiolysis and to allow for increased starting radioactivity. The purification and final product formulation use a custom-built, automated solid phase extraction (SPE) module that is designed to quickly complete the purification and product formulation while reducing radiolytic decomposition. The combination of improved synthesis, automated purification and formulation, and the use of radiolytic stabilizers has allowed for an increased starting activity of larger batches of [¹⁸F]DK222. This characteristic ultimately provides for the improved distribution of [¹⁸F]DK222.

More particularly, in some embodiments, the presently disclosed subject matter provides a method for preparing an ¹⁸F-labeled imaging agent comprising a conjugate of a peptide having a binding specificity for programmed death ligand 1 (PD-L1) and a chelating moiety, and optionally a linker, wherein the linker, when present connects the peptide and the chelating moiety, and when the linker is absent, the chelating moiety is attached directly to the peptide through a primary amine of an amino acid of the peptide, the method comprising: (a) providing or preparing a peptide conjugate precursor solution comprising a conjugate of a peptide having a binding specificity for programmed death ligand 1 (PD-L1) and a chelating moiety, and optionally a linker; (b) providing or preparing an anion exchange cartridge comprising [¹⁸F] ions; (c) adding an aluminum chloride solution, acetonitrile, ascorbic acid, and the peptide precursor solution to a reaction vessel; (d) eluting the anion exchange cartridge to release the [¹⁸F] ions into the reaction vessel; (e) heating the reaction vessel for a period of time; (f) cooling the reaction vessel; (g) adding ascorbic acid to the cooled reaction vessel to form a solution comprising the ¹⁸F-labeled imaging agent; (h) eluting the solution comprising the ¹⁸F-labeled imaging agent through a solid phase extraction cartridge; (i) washing the solid phase extraction cartridge with ascorbic acid; (j) eluting the solid phase extraction cartridge with an alkyl alcohol followed by sodium chloride for injection, USP with (+)-sodium L-ascorbate, through a sterilizing filter; and (k) collecting the ¹⁸F-labeled imaging agent.

In some embodiments, the peptide having a binding specificity for PD-L1 interacts with amino acids Y56, E58, A113, M115, and Y123 of PD-L1. In some embodiments, the peptide that interacts with PD-L1 is the peptide WL12. The peptide WL12 may have the amino acid sequence of Cyclo-(-Ac-Tyr-NMeAla-Asn-Pro-His-Leu-Hyp-Trp-Ser-Trp(methyl)-NMeNle-N MeNle-Lys-Cys-)-Gly-NH2 (SEQ ID NO.: 1). In some embodiments, WL12 may interact with four amino acids of PD-L1. In particular embodiments, WL12 may interact with amino acids Y56, E58, D61, and A113 of PD-L1. In some embodiments, WL12 may interact with five amino acids of PD-L1. In particular embodiments, WL12 may interact with amino acids Y56, E58, A113, M115 and Y123 of PD-L1. In other embodiments, the peptide that interacts with PD-L1 is DK221. The peptide DK221 may have the amino acid sequence of Cyclo-(-Ac-Tyr-NMeAla-Asn-Pro-His-Glu-Hyp-Trp-Ser-Trp(Carboxymethyl)-NMeN-le-N MeNle-Lys-Cys-)-Gly-NH2 (SEQ ID NO.: 2). In some embodiments, DK221 may interact with four amino acids of PD-L1. In particular embodiments, DK221 may interact with amino acids Y56, E58, D61, and A113 of PD-L1. In some embodiments, DK221 may interact with five amino acids of PD-L1. In particular embodiments, DK221 may interact with amino acids Y56, E58, A113, M115 and Y123 of PD-L1. In other embodiments, the peptide that interacts with PD-L1 is DK222. In some embodiments, DK222 may interact with four amino acids of PD-L1. In particular embodiments, DK222 may interact with amino acids Y56, E58, D61, and A113 of PD-L1. In some embodiments, DK222 may interact with five amino acids of PD-L1. In particular embodiments, DK222 may interact with amino acids Y56, E58, A113, M115 and Y123 of PD-L1.

In some embodiments, the peptide having a binding specificity for PD-L1 may have at least 80% sequence identity to SEQ ID NO.: 1. The peptide having a binding specificity for PD-L1 may have at least 80% sequence identity to SEQ ID NO.: 2. The peptide having a binding specificity for PD-L1 may have at least 85% sequence identity to SEQ ID NO.: 1. The peptide having a binding specificity for PD-L1 may have at least 85% sequence identity to SEQ ID NO.: 2. The peptide having a binding specificity for PD-L1 may have at least 90% sequence identity to SEQ ID NO.: 1. The peptide having a binding specificity for PD-L1 may have at least 90% sequence identity to SEQ ID NO.: 2. The peptide having a binding specificity for PD-L1 may have at least 95% sequence identity to SEQ ID NO.: 1. The peptide having a binding specificity for PD-L1 may have at least 95% sequence identity to SEQ ID NO.: 2. The peptide having a binding specificity for PD-L1 may have 100% sequence identity to SEQ ID NO.: 1. The peptide having a binding specificity for PD-L1 may have 100% sequence identity to SEQ ID NO.: 2.

The term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters, including default parameters for pairwise alignments.

In particular embodiments, the imaging agent comprises a compound of formula (I):

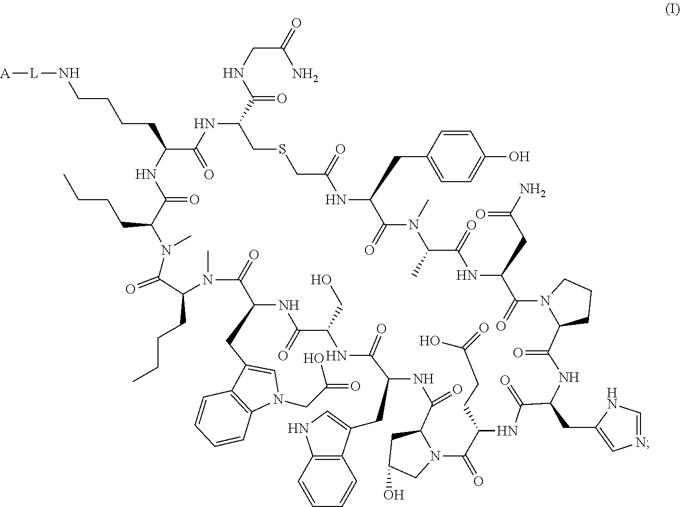

wherein:
L is a linker, which can be present or absent, and when present has the following general formula:

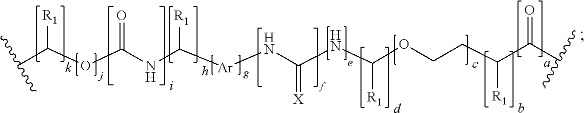

wherein:
X is S or O;
a, e, f, g, i, and j are each independently an integer selected the group consisting of 0 and 1;
b, d, h, and k are each independently an integer selected from the group consisting of 0, 1, 2, 3, 4, 5, 6, 7, and 8;
c is an integer having a range from 0 to 40;
each R₁ is H or —COOR₂, wherein R₂ is H or C₁-C₄ alkyl; Ar is substituted or unsubstituted aryl or heteroaryl; and
A is a chelating moiety.

In certain embodiments, the linker is selected from the group consisting of:

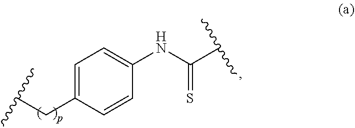

wherein p is an integer selected from 0, 1, 2, 3, and 4;

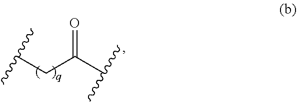

wherein q is an integer selected from the group consisting of 0, 1, 2, 3, 4, 5, 6, 7, and 8;

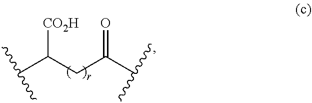

wherein r is an integer selected from the group consisting of 0, 1, 2, 3, 4, 5,6, 7, and 8;

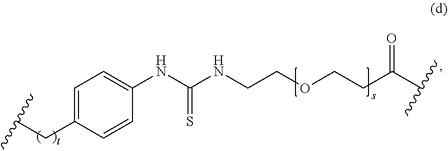

wherein s is an integer having a range from 1 to 40 and t is an integer selected from 0 or 1;

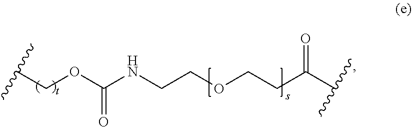

wherein s is an integer having a range from 1 to 40 and t is an integer selected from 0 or 1; and

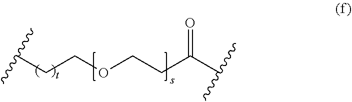

wherein s is an integer having a range from 1 to 40 and t is an integer selected from 0 or 1.

In particular embodiments, the chelating moiety is selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTA-tris(t-butyl)ester, DOTAGA-(t-butyl)₄, DOTA-di(t-butyl)ester, DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), DO3A-(t-butyl), DO3AM (2,2′,2″-(1,4,7,10-tetraazacyclododecane-1,4,7-triyl)triacetamide), p-NH₂-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NOTA(t-butyl)₂, NO2A (1,4,7-Triazacyclononane-1,4-bis(acetic acid)-7-(acetamide), NODA (1,4,7-triazacyclononane-1,4-diacetate); NODAGA (1,4,7-triazacyclononane, 1-glutaric acid-4,7-acetic acid), NODAGA(t-butyl)₃, NOTAGA (1,4,7-triazonane-1,4-diyl)diacetic acid), DFO (Desferoxamine), DTPA (2-[Bis [2-[bis(carboxymethyl)amino]ethyl]amino]acetic acid), DTPA-tetra(t-butyl)ester (diethylenetriamine-N,N,N″,N″-tetra-tert-butyl acetate-N′-acetic acid), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3,6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), BaBaSar, tris(hydroxypyridinone) (THP), THP(benzyl)₃, NOPO (3-(((4,7-bis((hydroxy(hydroxymethyl)phosphoryl)-methyl)-1,4,7-triazonan-1-yl)methyl)(hydroxy)phosphoryl)propanoic acid), TRAP (3,3′,3″-(((1,4,7-triazonane-1,4,7-triyl)tris(methylene))tris(hydroxyphosphoryl))-tripropanoic acid), p-NH₂-Bn-PCTA (3,6,9,15-Tetraazabicyclo[9.3.1]pentadeca-1(15),11,13-triene-4-S-(4-aminobenzyl)-3,6,9-triacetic acid), and biotin (5-[(3aS,4S,6aR)-2-oxohexahydro-1H-thieno[3,4-d]imidazol-4-yl]pentanoic acid).

In more particular embodiments, the chelating moiety is selected from the group consisting of:

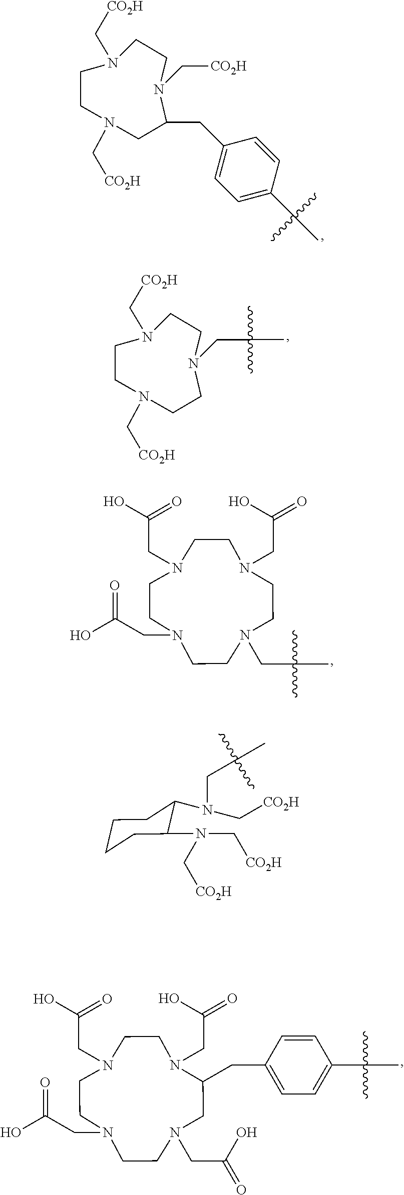 [SMILES: *c1ccc(CC2CN(CC(=O)O)CCN(CC(=O)O)CCN2CC(=O)O)cc1 ; *CN1CCN(CC(=O)O)CCN(CC(=O)O)CC1 ; *CN1CCN(CC(=O)O)CCN(CC(=O)O)CCN(CC(=O)O)CC1 ; *CN(CC(=O)O)C1CCCCC1N(CC(=O)O)CC(=O)O ; *c1ccc(CC2CN(CC(=O)O)CCN(CC(=O)O)CCN(CC(=O)O)CCN2CC(=O)O)cc1]

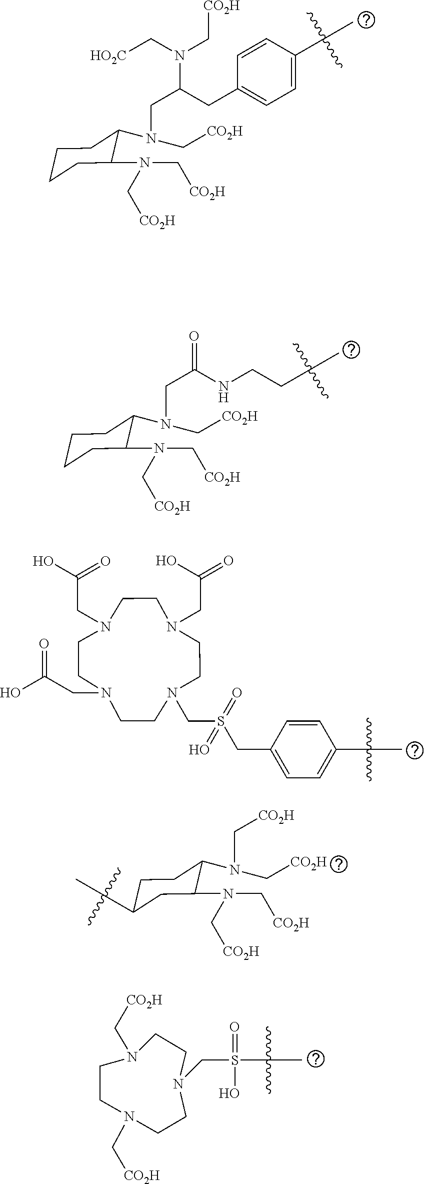

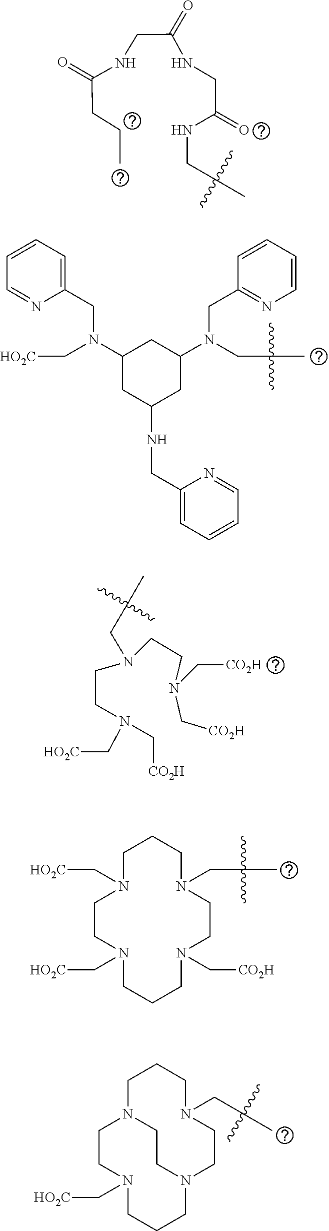 [SMILES: *CNC(=O)CNC(=O)CNC(=O)CCC ; *CN(Cc1ccccn1)C1CC(NCc2ccccn2)CC(N(CC(=O)O)Cc2ccccn2)C1 ; *CN(CCN(CC(=O)O)CC(=O)O)CCN(CC(=O)O)CC(=O)O ; *CN1CCCN(CC(=O)O)CCN(CC(=O)O)CCCN(CC(=O)O)CC1 ; *CN1CCCN2CCN(CCCN(CC(=O)O)CC2)CC1]

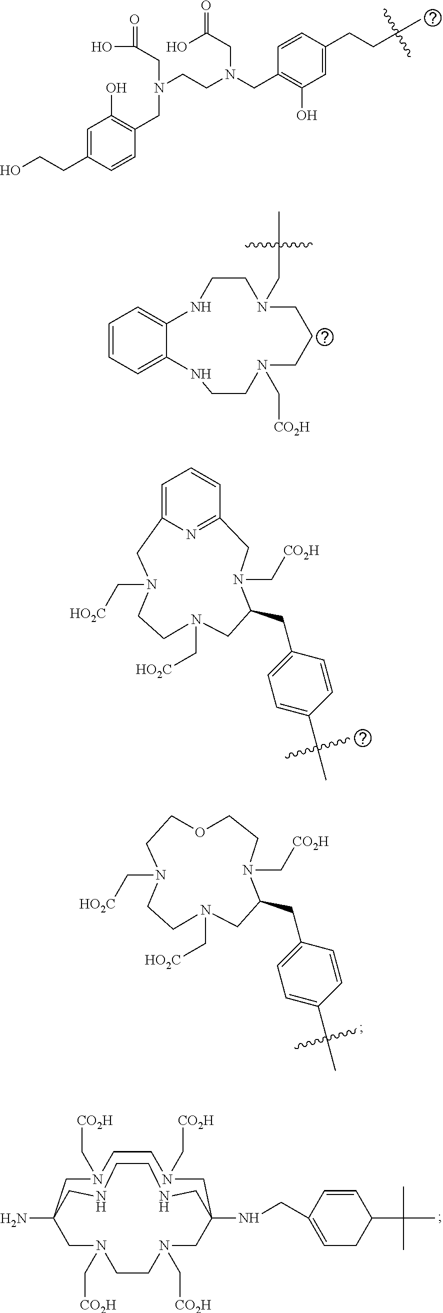 [SMILES: *CCc1ccc(CN(CCN(CC(=O)O)Cc2ccc(CCO)cc2O)CC(=O)O)c(O)c1 ; *CN1CCCN(CC(=O)O)CCNc2ccccc2NCC1 ; *c1ccc(C[C@H]2CN(CC(=O)O)CCN(CC(=O)O)Cc3cccc(n3)CN2CC(=O)O)cc1 ; *c1ccc(C[C@H]2CN(CC(=O)O)CCN(CC(=O)O)CCOCCN2CC(=O)O)cc1 ; CC(C)(C)C1C=CC(CNC23CNCCNCC(N)(CN(CC(=O)O)CCN(CC(=O)O)C2)CN(CC(=O)O)CCN(CC(=O)O)C3)=CC1]

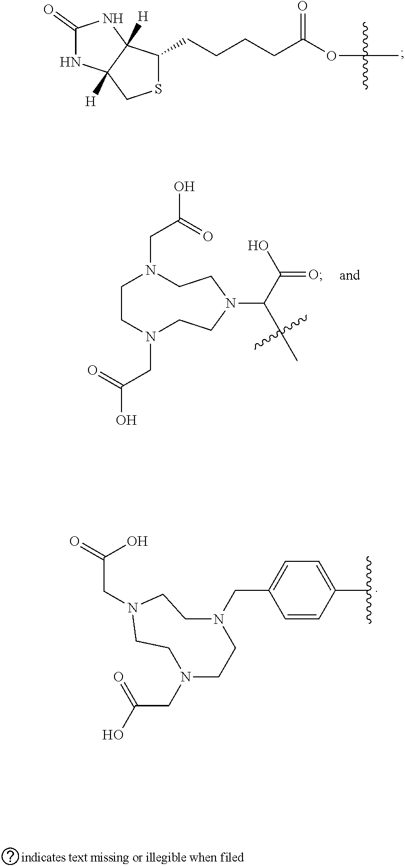 [SMILES: *OC(=O)CCCC[C@@H]1SC[C@@H]2NC(=O)N[C@@H]21 ; *C(C(=O)O)N1CCN(CC(=O)O)CCN(CC(=O)O)CC1 ; *c1ccc(CN2CCN(CC(=O)O)CCN(CC(=O)O)CC2)cc1]

In yet more particular embodiments, the chelating moiety is selected from the group consisting of DOTA (1,4,7,10-tetraazacyclododecane-1,4,7,10-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane, 1-glutaric acid-4,7-acetic acid), and biotin (5-[(3aS,4S,6aR)-2-oxohexahydro-1H-thieno[3,4-d]imidazol-4-yl]pentanoic acid).

In certain embodiments, the imaging agent is selected from the group consisting of:

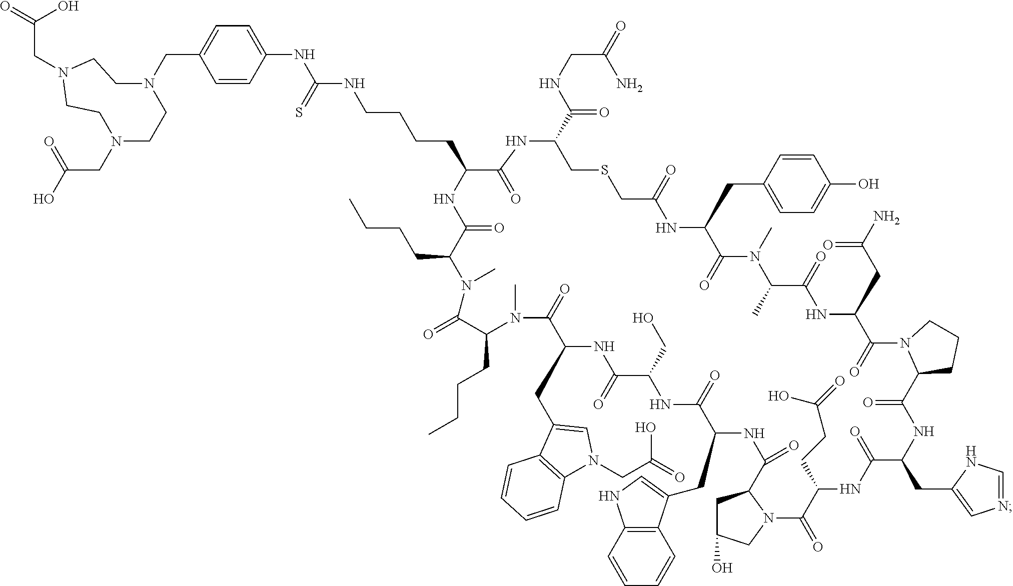 [SMILES: CCCC[C@H]1C(=O)N(C)[C@@H](CCCC)C(=O)N[C@@H](CCCCNC(=S)Nc2ccc(CN3CCN(CC(=O)O)CCN(CC(=O)O)CC3)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

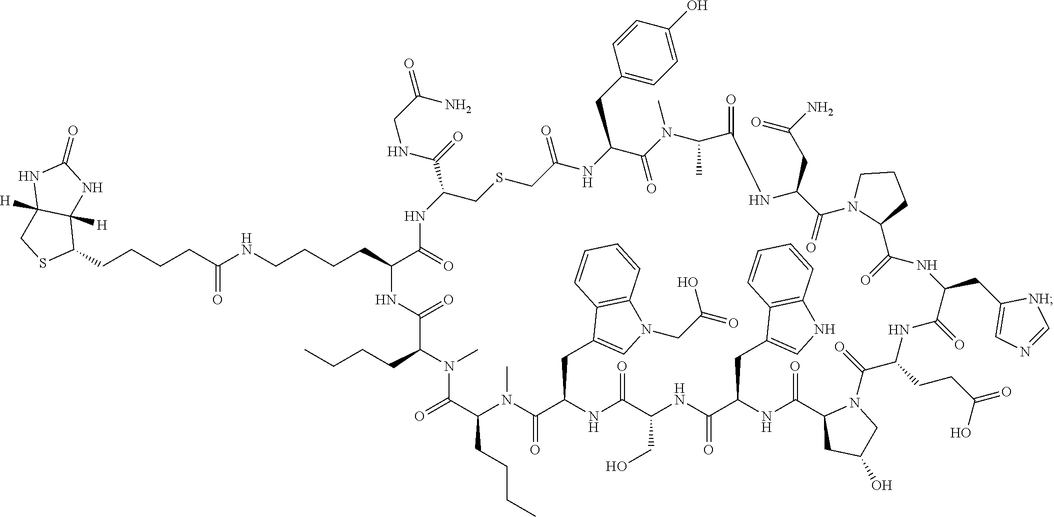 [SMILES: CCCC[C@H]1C(=O)N(C)[C@@H](CCCC)C(=O)N[C@@H](CCCCNC(=O)CCCC[C@@H]2SC[C@@H]3NC(=O)N[C@@H]32)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@H](CCC(=O)O)C(=O)N2C[C@H](O)C[C@H]2C(=O)N[C@H](Cc2c[nH]c3ccccc23)C(=O)N[C@H](CO)C(=O)N[C@H](Cc2cn(CC(=O)O)c3ccccc23)C(=O)N1C]

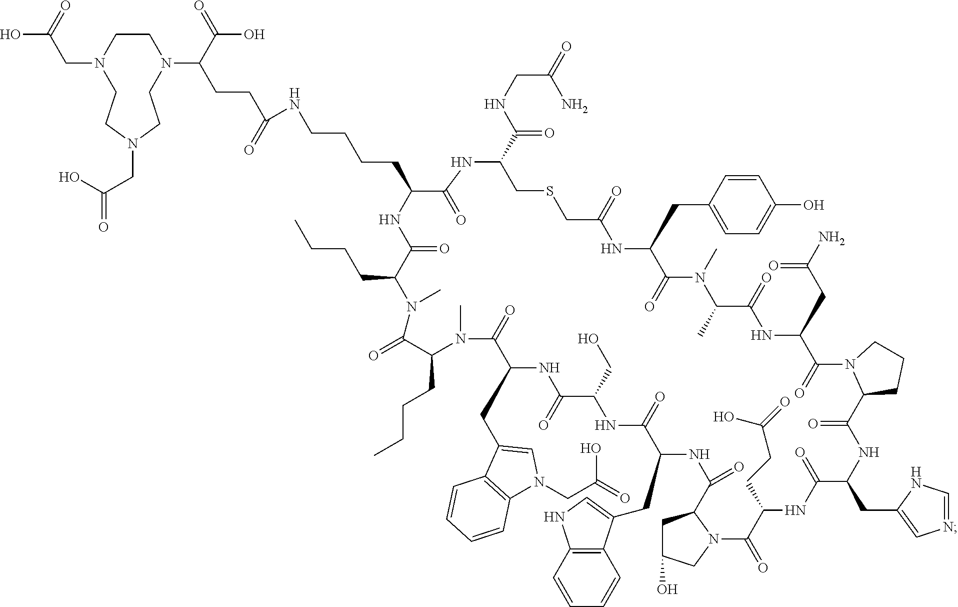 [SMILES: CCCC[C@H]1C(=O)N(C)[C@@H](CCCC)C(=O)N[C@@H](CCCCNC(=O)CCC(C(=O)O)N2CCN(CC(=O)O)CCN(CC(=O)O)CC2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

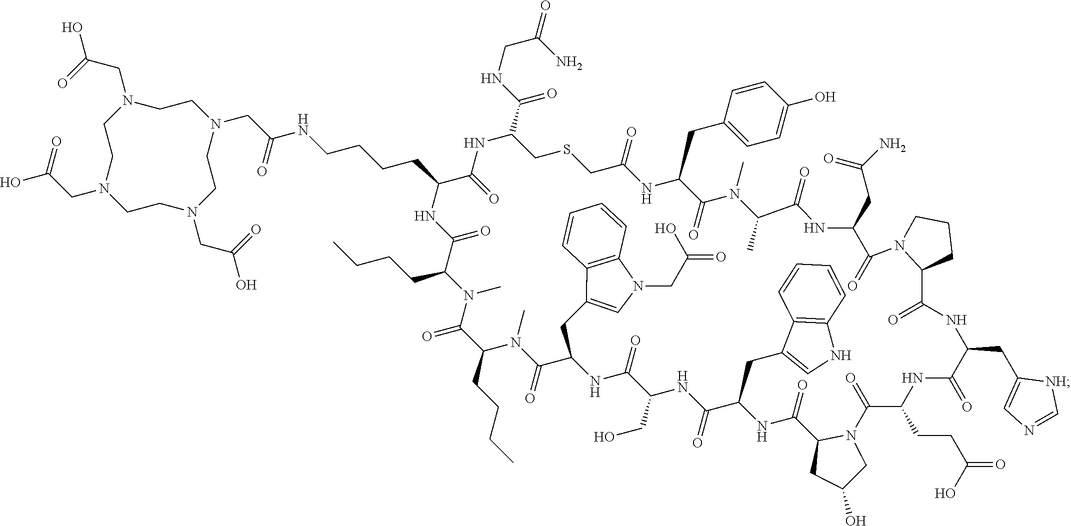

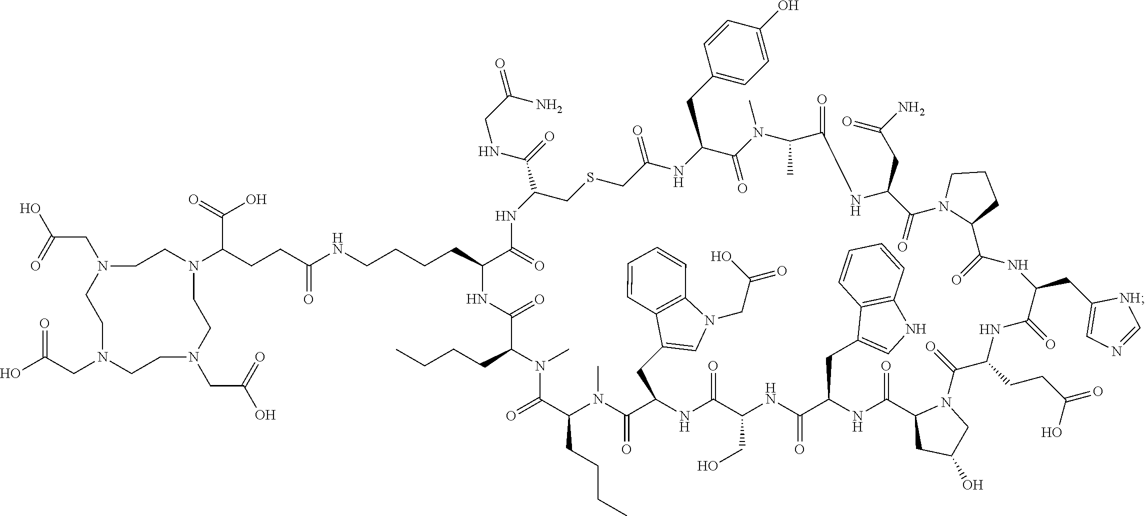

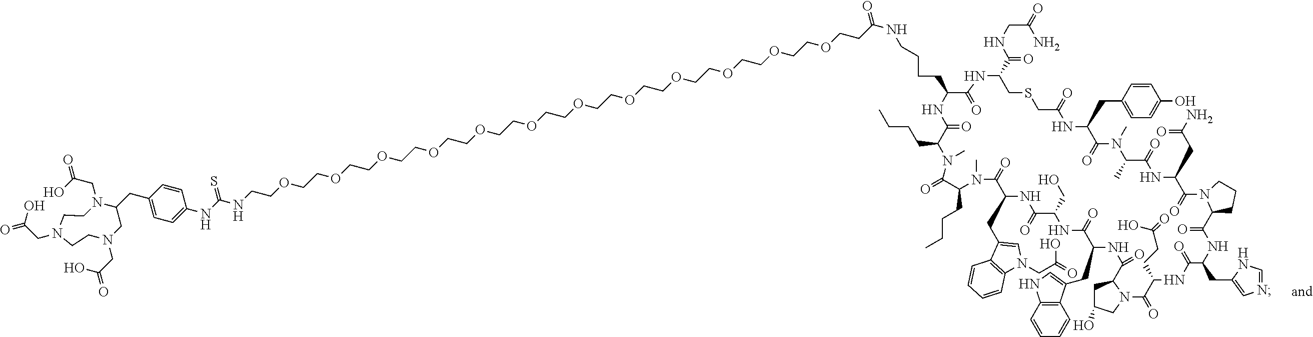 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCNC(=S)Nc2ccc(CC3CN(CC(=O)O)CCN(CC(=O)O)CCN3CC(=O)O)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

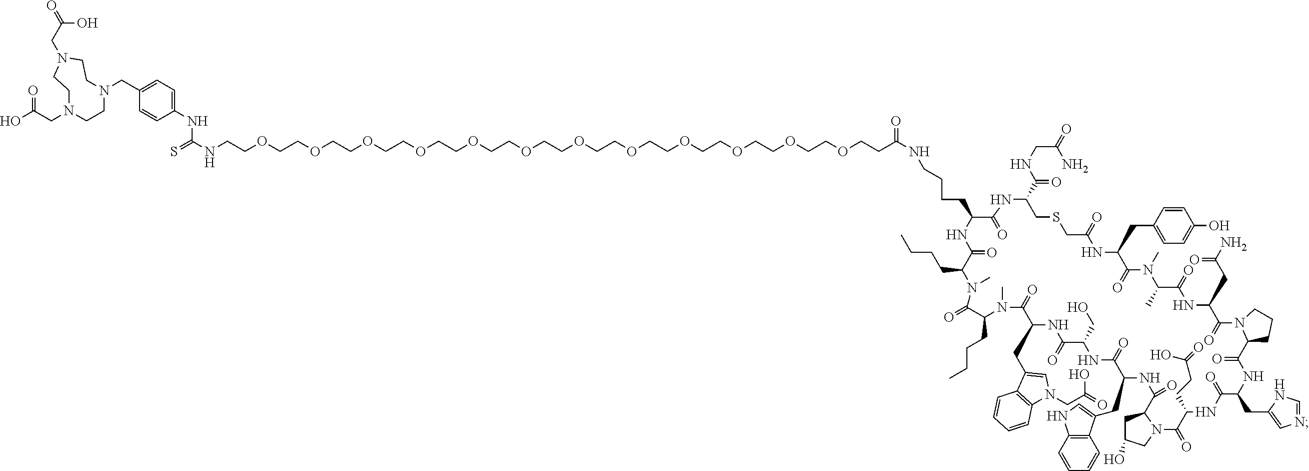 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCNC(=S)Nc2ccc(CN3CCN(CC(=O)O)CCN(CC(=O)O)CC3)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

wherein the chelating moiety of each compound comprises Al[¹⁸F].

In more certain embodiments, the ¹⁸F-labeled imaging agent is [¹⁸F]DK222 having the following chemical structure:

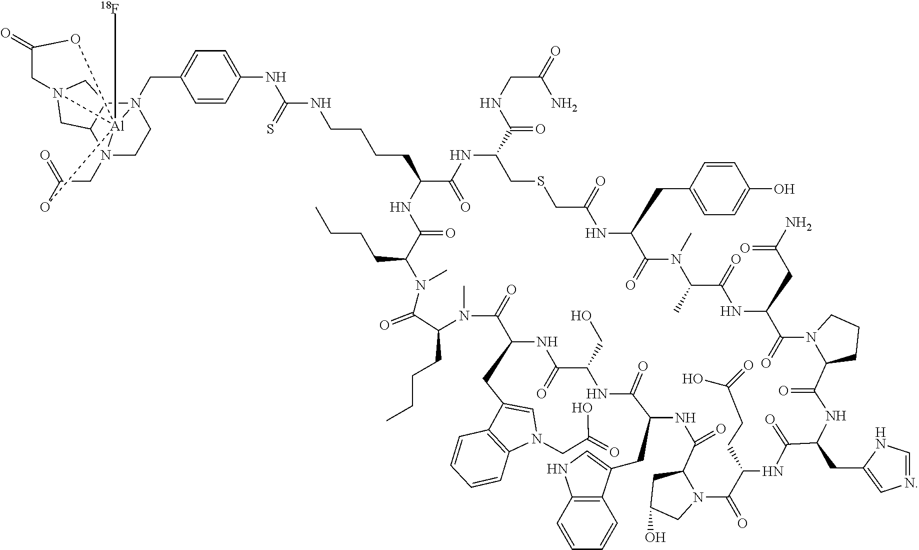

In some embodiments, the aluminum chloride solution comprises an aluminum chloride hexahydrate solution in ascorbic acid. In some embodiments, the ascorbic acid at each occurrence comprises a 5-M ascorbic acid aqueous solution. In some embodiments, the anion exchange cartridge is eluted with a base. In some embodiments, the base is potassium acetate.

In some embodiments, the reaction vessel is heated to about 110° C.±20° C., including 90° C., 95° C., 100° C., 105° C., 110° C., 115° C., 120° C., 125° C., and 130° C. In certain embodiments, the reaction vessel is heated for about 10 minutes. In certain embodiments, the reactive vessel is cooled to less than about 50° C., including about 25° C., 30° C., 35° C., 40° C., 45° C., and 49° C.

In some embodiments, the solid phase extraction cartridge comprises a C18 or tC18 sorbent.

In some embodiments, the alkyl alcohol is selected from the group consisting of methanol, ethanol, propyl alcohol, isopropyl alcohol, and butanol. In particular embodiments, the alkyl alcohol is ethanol.

In some embodiments, the sterilizing filter comprises a polytetrafluoroethylene membrane.

In particular embodiments, one or more of steps (a)-(k) are automated.

In some embodiments, the ¹⁸F-labeled imaging agent meets one or more quality control criteria selected from the group consisting of: (a) a clear, colorless solution with no visible particulate matter; (b) a yield not less than 20 mCi of the ¹⁸F-labeled imaging agent; (c) a pH in the range of 3.5 to 5.5; (d) the sterilizing filter has a greater than or equal to 13 psi bubble-point test; (e) a radionuclidic identity and purity in the range of 105-115 minutes for half-life measurement; (f) a radiochemical purity of 90% or greater for the ¹⁸F-labeled imaging agent; (g) a specific activity greater than 250 mCi/μmole; (h) less than 10% ethanol and less than 273 parts per million acetonitrile residual solvent; and (i) bacterial endotoxins equal to or less than eleven USP endotoxin units per mL. As used herein, the term “endotoxin” means a toxin that is present inside a bacterial cell and is released when the cell disintegrates. It is sometimes responsible for the characteristic symptoms of a disease, e.g., in botulism. In certain embodiments, the method further comprises inoculating the ¹⁸F-labeled imaging agent at the end of each production day and monitoring for 14 days for visible growth of microorganisms.

In some embodiments, the method comprises a starting radioactivity of the [¹⁸F] ions comprising the anion exchange cartridge having a range from about 100 mCi to greater than about 1,000 mCi, including 100, 150, 200, 250, 300, 350, 400, 450, 500, 550, 600, 650, 700, 750, 800, 850, 900, 950, and 1000 mCi, and in some embodiments up to 10,000 mCi, including 1,100, 2,000, 3,000, 4,000, 5,000, 6,000, 7,000, 8,000, 9,000, and 10,000 mCi. In some embodiments, the ¹⁸F-labeled imaging agent has a radioactivity having a range from about 50 mCi to greater than about 500 mCi, including about 50, 60, 70, 80, 90, 100, 150, 200, 250, 300, 350, 400, 450, and 500 mCi, and in some embodiments, up to 5,000 mCi, including 600, 700, 800, 900, 1,000, 1,100, 1,200, 1,300, 1,400, 1,500, 1,600, 1,700, 1,800, 1,900, 2,000, 3,000, 4,000, and 5,000 mCi. In some embodiments, the ¹⁸F-labeled imaging agent has a yield at the end of synthesis (EOS) having a range from about 30% to about 60%, including about 30%, 35%, 40%, 45%, 50%, 55%, and 60% In some embodiments, the ¹⁸F-labeled imaging agent has a specific activity having a range from about 2,000 mCi/μmole to greater than about 25,000 mCi/μmole, including about 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, and 25,000 mCi/μmole. In some embodiments, the ¹⁸F-labeled imaging agent has an effective specific activity having a range from about 900 mCi/μmole to about 8,000 mCi/μmole, including about 900, 1,000, 2,000, 3,000, 4,000, 5,000, 6,000, 7,000, and 8,000 mCi/μmole. In some embodiments, the ¹⁸F-labeled imaging agent has a radiochemical activity having a range from about 90% to about 100%, including about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, and 100%.

In some embodiments, the presently disclosed subject matter provides a kit for detecting Programmed Death Ligand 1 (PD-L1), the kit comprising a peptide conjugate precursor solution comprising a conjugate of a peptide having a binding specificity for programmed death ligand 1 (PD-L1) and a chelating moiety, and optionally a linker.

In certain embodiments, the kit further comprises an anion exchange cartridge comprising [¹⁸F] ions. In certain embodiments, the kit further comprises a solid phase extraction cartridge. In certain embodiments, the kit further comprises one or more solvents or reagents necessary for preparing an ¹⁸F-labeled imaging agent by the presently disclosed methods.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### [¹⁸F]DK222 Synthesis

A solution of 0.5-M ascorbic acid is prepared and attached to a solid phase extraction (SPE) module. Sodium chloride for injection USP with (+)-sodium L-ascorbate is prepared and attached to the SPE module. A tC18 Sep-Pak® plus cartridge (Waters Corporation, Milford, Massachusetts, USA) is primed with 10 mL of 0.5-M ascorbic acid and attached to the SPE module. The tC18 Sep-Pak® plus cartridge includes a silica-based bonded phase with strong hydrophobicity with trifunctional bonding chemistry giving it an increased hydrolytic stability.

A 1.78-mM solution of aluminum chloride hexahydrate is prepared using 0.5-M ascorbic acid. A 0.425-mM solution of DK-NODA is prepared using an 80/20 ratio of acetonitrile/0.5-M ascorbic acid. A 102-mM solution of potassium acetate is prepared with high purity water.

A reaction vial, washed with nitric acid, rinsed with water followed by ethanol (ABS), then dried at a temperature greater than 80° C. is prepared for the synthesis. To the cleaned vial, 45 microliters of the aluminum chloride solution is added, followed by 150 microliters of acetonitrile, 50 microliters of 0.5-M ascorbic acid, and 200 microliters of the DK-NODA precursor solution. The precursor mixture can be used immediately or evaporated to dryness for later use. To the evaporated precursor mixture, the addition of 445 microliters of 70/30 acetonitrile/0.5-M ascorbic acid is required.

[¹⁸F]fluoride ions are trapped on an anion exchange cartridge. The anion exchange cartridge is eluted into the prepared reaction vial with the solution of potassium acetate to release the [¹⁸F]fluoride ion from the anion exchange cartridge. The reaction vial is heated at about 110° C.±20° C. for 10 minutes. After heating the reaction vial is cooled to less than 50° C.

The purification and reformulation are completed by the automated SPE module. The SPE module adds to the reaction vial 4.5 mL of 0.5-M ascorbic acid. The SPE module aspirates the diluted solution into the glass syringe, then elutes the solution through a tC18 Sep-Pak® plus cartridge. The SPE module then washes the tC18 Sep-Pak® plus cartridge with 5 mL of 0.5-M ascorbic acid. The SPE module then elutes the tC18 Sep-Pak® plus cartridge with 1 mL of ethanol (ABS) followed by 14 mL of sodium chloride for injection, USP with (+)-sodium L-ascorbate into the final product vial via a Millipore 0.2 μm FG sterilizing filter, for example, a 25-mm diameter filter comprising a hydrophobic polytetrafluoroethylene (PTFE) membrane having a 0.2-μm pore size and which has been sterilized, for example, with ethylene oxide.

### Example 2

### Quality Control

In certain embodiments, [¹⁸F]DK222 quality control consists of ten requirements to be met or exceeded before the product is to be released for use. These quality control requirements include, but are not limited to: appearance shall be clear, colorless solution with no visible particulate matter; yield shall not be less than 20 mCi of [¹⁸F]DK222; the pH shall be in the range of 3.5-5.5; the sterile Millipore FG filter shall have a greater than or equal to 13 psi bubble-point test requirement; the radionuclidic identity and purity shall fall in the range of 105-115 minutes for half-life measurement; the radiochemical purity shall be 90% or greater for [¹⁸F]DK222; the specific activity shall be greater than 250 mCi/μmole; the residual solvent analysis shall ensure ethanol is less than 10% and acetonitrile is less than 273 parts per million; and the bacterial endotoxins shall be required to be equal to or less than eleven USP endotoxin units per mL. In addition to the above ten quality control tests, sterility testing shall be inoculated at the end of each day of production and shall be monitored for 14 days with visible growth.

The HPLC quality control to determine radiochemical purity and specific activity of [¹⁸F]DK222 is preformed using a USP designation L1 HPLC column (Phenomenex Luna C18(2) 4.6-mm×150-mm, 5 μm). The HPLC conditions are gradient HPLC method, mobile phase “A” consisting of 30% acetonitrile:70% water:0.3% trifluoroacetic acid. Mobile phase “B” consisting of 100% acetonitrile:0.3% trifluoroacetic acid. The flow rate of 1.5 mL/min is used with a UV wavelength of 254 nm.

TABLE 1
Analytical HPLC Gradient

| | | | Flow |
|---|---|---|---|
| Time | A (%) | B (%) | (mL/min) |
| 0.00 | 100 | 0.0 | 1.5 |
| 0.20 | 100 | 0.0 | 1.5 |
| 10.00 | 75.5 | 24.5 | 1.5 |

### Example 3

### Radiolytic Stabilizers

[¹⁸F]DK222 yield and radiochemical purity was greatly improved by the use of ascorbic acid and (+)-sodium L-Ascorbate in the synthesis, purification, and formulation. Previously, the synthesis used acetic acid-sodium acetate buffer at pH 4.2, but testing showed that this buffer limited the amount of starting radioactivity to around 100 mCi to achieve a radiochemical pure [¹⁸F]DK222. Changing the synthesis to incorporate 0.5-M ascorbic acid at pH 2.2 allowed for increasing the starting radioactivity and thus being able to make larger amounts of [¹⁸F]DK222 and still achieve greater than 90% radiochemical purity. The use of 0.5-M ascorbic acid in the purification step was of critical importance to reduce radiolysis during the purification process. Likewise, using sodium ascorbate in the formulation of [¹⁸F]DK222 allowed for increased starting radioactivity to achieve larger batches sizes.

### Example 4

### [¹⁸F]DK222 Automated Purification and Formulation

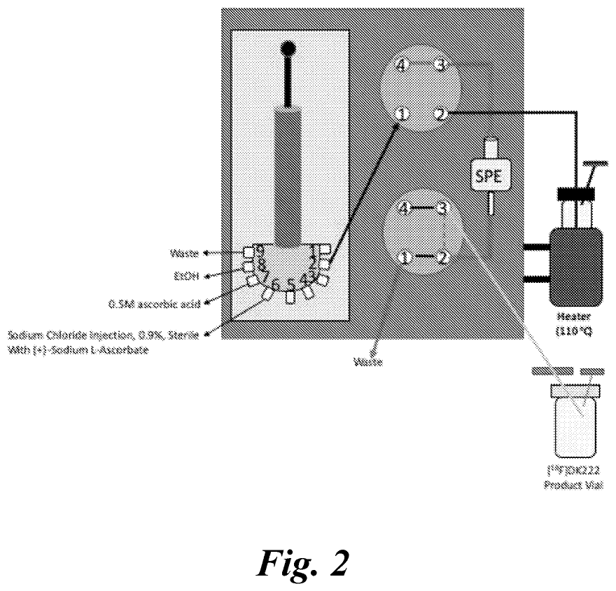

Automated purification and reformulation are accomplished by modifying a JHU PET Radiochemistry designed and built solid phase extraction (SPE) module to include a heating block. See FIG. 2. The SPE module is controlled by National Instruments ethernet based module I/O controllers using NI Lab View software. Using NI Labview, a custom, automated program was written to allow the user an intuitive interface to initiate the purification and formulation of [¹⁸F]DK222. This automated purification and formulation greatly speeds up the process over the previous manual method. Radiolysis of [¹⁸F]DK222 has been greatly reduced because of the more rapid process of the automated SPE module.

The combination of radiolytic stabilizers with a more rapid automated purification and formulation have allowed for starting radioactivity to exceed 1,000 mCi, resulting in batches of [¹⁸F]DK222 exceeding 500 mCi while maintaining greater than 90% radiochemical purity.

### Example 5

### Automated Synthesis of DK222 With a Commercial PET Synthesis Module

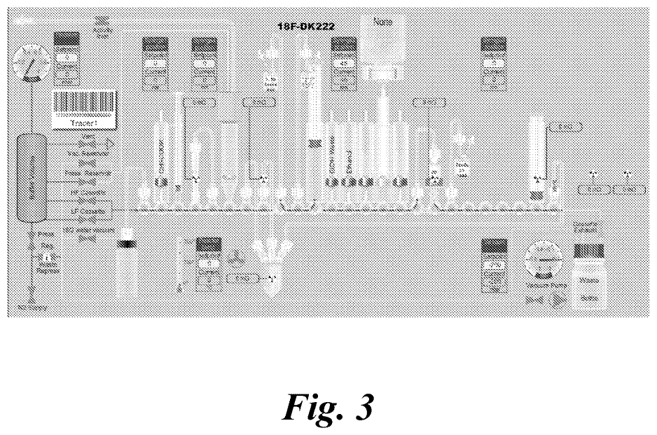

Referring now to FIG. 3, is a schematic diagram for the automated synthesis of DK222 with a commercial apparatus, e.g., a FASTlab™ 2 PET synthesizer (GE Healthcare, United States). Using this apparatus, a [¹⁸F]DK222 FASTlab cassette is assembled with the vial, reagent and cartridges additions: potassium acetate solution (position 2), 45-mg PS-HCO₃-anion exchange cartridge (position 4), 0.5-M ascorbic acid solution (position 9), sodium chloride for injection, USP with (+)-sodium L-ascorbate solution (position 10), empty vial (position 12), ethanol (position 13), tC18 Sep-Pak® plus cartridge (Waters Corporation, Milford, Massachusetts, USA) (position 18), product vial (position 19).

A 1.78-mM solution of aluminum chloride hexahydrate is prepared using 0.5-M ascorbic acid. A 0.425-mM solution of DK-NODA is prepared using an 80/20 ratio of acetonitrile/0.5-M ascorbic acid. A 102-mM solution of potassium acetate is prepared with high purity water.

To the FASTlab reaction vessel, 45 microliters of the aluminum chloride solution is added, followed by 150 microliters of acetonitrile, 50 microliters of 0.5-M ascorbic acid, and 200 microliters of the DK-NODA precursor solution. The reaction vessel is connected to the FASTlab cassette and the vessel is placed in the reaction heater.

The FASTlab synthesis sequence is started. [¹⁸F]fluoride is delivered to the FASTlab collection vessel and the sequence transfer the fluoride to the 45-mg PS-HCO₃-anion exchange cartridge. The [¹⁸F]fluoride is released from the cartridge with the solution of potassium acetate, transferring to the reaction vessel. The reaction vessel is heated 110° C.±20° C. for 10 minutes, before being cooled.

The purification and reformulation is completed by the FASTlab module. To the reaction vessel, 4.5 mL of 0.5-M ascorbic acid is added. The diluted reaction solution is transferred through a tC18 Sep-Pak® plus cartridge (position 18). The tC18 Sep-Pak® plus cartridge is washed with 5 mL of 0.5-M ascorbic acid. Then the tC18 Sep-Pak® plus cartridge is eluted with 1 mL of ethanol (ABS) followed by 14 mL of sodium chloride for injection, USP with (+)-sodium L-ascorbate into the final product vial via a Millipore 0.2-μm FG sterilizing filter, for example, a 25-mm diameter filter comprising a hydrophobic polytetrafluoroethylene (PTFE) membrane having a 0.2-μm pore size and which has been sterilized, for example, with ethylene oxide.

### Example 6

### Improved Synthesis Results

TABLE 2
Representative Synthesis Results

| | | Final | Yield % | | Effective | |
|---|---|---|---|---|---|---|
| | Starting | Product | at End of | Specific | Specific | |
| | radioactivity | [¹⁸F]DK222 | Synthesis | activity | activity | Radiochemical |
| Sample | (mCi) | (mCi) | (EOS) | mCi/μmole | mCi/μmole | Purity |
| 1 | 173 | 90.2 | 52.1 | 3,442 | 1,327 | 100% |
| 2 | 115 | 64.7 | 56.3 | 2,250 | 911 | 100% |
| 3 | 140 | 71.4 | 51.0 | 3.379 | 1227 | 100% |
| 4 | 172 | 74.9 | 43.5 | 2,857 | 1429 | 100% |
| 5 | 322 | 110.8 | 34.4 | 8,250 | 2,992 | 100% |
| 6 | 620 | 287.3 | 46.3 | 15,500 | 5,446 | 99.7% |
| 7 | 565 | 256 | 45.3 | 13,222 | 3,757 | 100% |
| 8 | 624 | 326 | 52.2 | 12,611 | 4,018 | 100% |
| 9 | 1096 | 502 | 45.8 | 25,715 | 7,959 | 99.7% |
| 10 | 850 | 453.5 | 53.3 | 16,474 | 6,078 | 99.4% |
| 11 | 794 | 289.5 | 36.5 | 16,167 | 4,675 | 100% |
| Average | 497 | 229.7 | 47.0 | 10,590 | 3,620 | 99.9% |

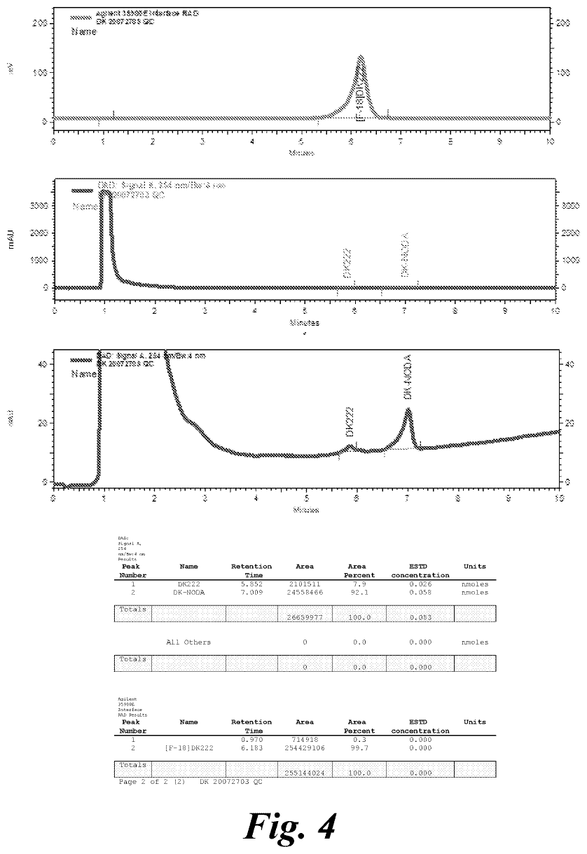

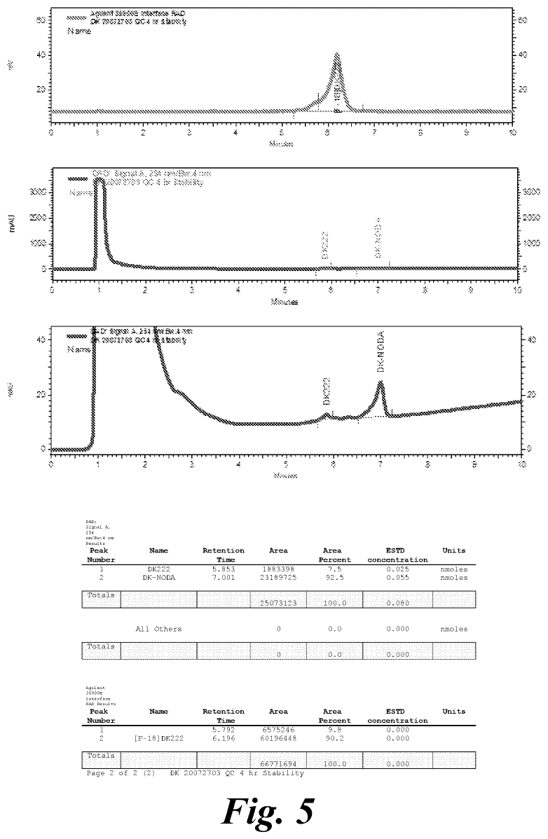

Representative HPLC chromatograms demonstrating the quality control and stability of sample #9 are shown in FIG. 4 and FIG. 5, respectively.

### Example 7

### Comparison of Synthesis of DK222 With Commercial FASTlab Module vs Manual Synthesis With Automated SPE Purification Using a Custom-Built SPE System

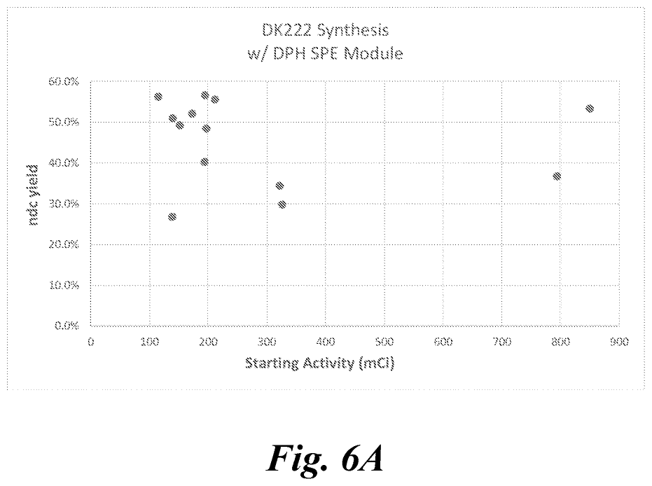

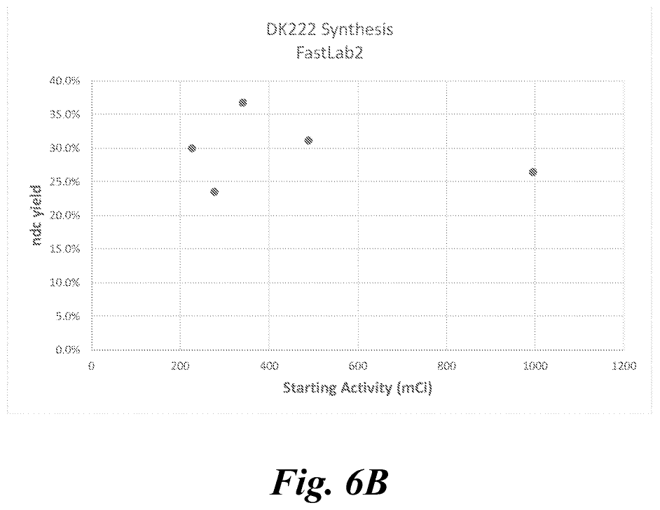

Referring now to Table 3 and FIG. 6A and FIG. 6B is a comparison of the synthesis of DK222 with a commercial FASTlab 2 module vs. manual synthesis with an automated SPE purification using a custom-built SPE system. More particularly, FIG. 6A is a plot of the non-decay corrected (ndc) yield versus starting activity (mCi) for the synthesis of DK222 with a custom-built SPE module. FIG. 6B is a plot of the non-decay corrected (ndc) yield versus starting activity (mCi) for the synthesis of DK222 with a commercial FASTlab 2 module.

TABLE 3
Comparison of DK222 Synthesis with Custom-
Built SPE Module vs. FASTlab 2

| DK222 validation runs | | Starting | DK222 | | Effective SA |
|---|---|---|---|---|---|
| Run # | Module | mCi | mCi | % yield | mCi/umole |
| 1 | DPH | 173 | 90.2 | 52.1% | 1,327 |
| | Module | | | | |
| 2 | DPH | 115 | 64.7 | 56.3% | 911 |
| | Module | | | | |
| 3 | DPH | 140 | 71.4 | 51.0% | 1,227 |
| | Module | | | | |
| 4 | DPH | 152 | 74.9 | 49.3% | 1,429 |
| | Module | | | | |
| 5 | DPH | 322 | 110.8 | 34.4% | 2,992 |
| | Module | | | | |
| 6 | DPH | 850 | 453.6 | 53.4% | 6,078 |
| | Module | | | | |
| 7 | DPH | 794 | 291.5 | 36.7% | 4,675 |
| | Module | | | | |
| 8 | DPH | 326 | 97.0 | 29.8% | 1,593 |
| | Module | | | | |
| 9 | DPH | 139 | 37.2 | 26.8% | 826 |
| | Module | | | | |
| 10 | DPH | 195 | 110.5 | 56.7% | 1,753 |
| | Module | | | | |
| 11 | DPH | 197 | 95.4 | 48.4% | 1,464 |
| | Module | | | | |
| 12 | DPH | 212 | 117.9 | 55.6% | 1,807 |
| | Module | | | | |
| 13 | DPH | 194 | 78.1 | 40.3% | 1,106 |
| | Module | | | | |
| Average | | 293 | 130.2 | 45.4% | 2,091 |
| FastLab 2 runs | | | | | |
| 1 | 1st | 277 | 65.1 | 23.5% | 972 |
| | attempt | | | | |
| 2 | 2nd | 341 | 125.3 | 36.7% | 2,116 |
| | attempt | | | | |
| 3 | FastLab | 489 | 152.2 | 31.1% | 2,246 |
| 4 | FastLab | 226 | 67.7 | 30.0% | 1,172 |
| 5 | FastLab | 995 | 263.0 | 26.4% | 4,471 |
| 6 | FastLab | 280 | 89.7 | 32.0% | 1,289 |
| Average | | 435 | 127.2 | 30.0% | 2044 |

### REFERENCES

All publications, patent applications, patents, and other references mentioned in the specification are indicative of the level of those skilled in the art to which the 5 presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Davis, A. A. and Patel, V. G. The role of PD-L1 expression as a predictive biomarker: an analysis of all US Food and Drug Administration (FDA) approvals of immune checkpoint inhibitors. J Immunother Cancer 7, 278 (2019).
McLaughlin, J. et al. Quantitative Assessment of the Heterogeneity of PD-L1 Expression in Non-Small-Cell Lung Cancer. JAMA Oncol 2, 46-54 (2016).
Gandini, S., Massi, D. and Mandala, M. PD-L1 expression in cancer patients receiving anti PD-1/PD-L1 antibodies: A systematic review and meta-analysis. Crit Rev Oncol Hematol 100, 88-98 (2016).
Jacobson, O., Kiesewetter, D. O. and Chen, X. Fluorine-18 radiochemistry, labeling strategies and synthetic routes. Bioconjug Chem 26, 1-18 (2015).
McBride, W. J. et al. Improved ¹⁸F labeling of peptides with a fluoride-aluminum-chelate complex. Bioconjug Chem 21, 1331-1340 (2010).
Wan, W. et al. First experience of ¹⁸F-alfatide in lung cancer patients using a new lyophilized kit for rapid radiofluorination. J Nucl Med 54, 691-698 (2013).
D'Souza, C. A., McBride, W. J., Sharkey, R. M., Todaro, L. J. and Goldenberg, D. M. High-yielding aqueous ¹⁸F-labeling of peptides via Al¹⁸F chelation. Bioconjug Chem 22, 1793-1803 (2011).
Lutje, S. et al. In Vitro and In Vivo Characterization of an ¹⁸F-AIF-Labeled PSMA Ligand for Imaging of PSMA-Expressing Xenografts. J Nucl Med 60, 1017-1022 (2019).
Kumar, K. and Ghosh, A. ¹⁸F-AlF Labeled Peptide and Protein Conjugates as Positron Emission Tomography Imaging Pharmaceuticals. Bioconjug Chem 29, 953-975 (2018).
Liu, T. et al. Preclinical Evaluation and Pilot Clinical Study of Al¹⁸F-PSMA-BCH for Prostate Cancer PET Imaging. J Nucl Med 60, 1284-1292 (2019).
Yu, C. et al. ¹⁸F-Alfatide II PET/CT in healthy human volunteers and patients with brain metastases. Eur J Nucl Med Mol Imaging 42, 2021-2028 (2015).
Tshibangu, T. et al. Automated GMP compliant production of [¹⁸F]Al-NOTA-octreotide. EJNMMI Radiopharm Chem 5, 4 (2020).
Giglio, J., Zeni, M., Savio, E. and Engler, H. Synthesis of an Al¹⁸F radiofluorinated GLU-UREA-LYS(AHX)-HBED-CC PSMA ligand in an automated synthesis platform. EJNMMI Radiopharm Chem 3, 4 (2018).
Kersemans, K. et al. Automated radiosynthesis of Al[¹⁸F]PSMA-11 for large scale routine use. Appl Radiat Isot 135, 19-27 (2018).
Kumar D., et al., Fluorine-18-labeled peptide-based PET provides PD-L1-specific high contrast images, J Nucl Med 60 (supplement 1), 542 (2019).
Johns Hopkins, 2021, J2096—An Open-label, Single-center Clinical Trial of [¹⁸F]DK222 PET/CT for Imaging Patients with Non-Small-Cell Lung Cancer (NSCLC) who are Eligible for Treatment with Anti-PD-1 Monotherapy.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for preparing an ¹⁸F-labeled imaging agent comprising a conjugate of a peptide having a binding specificity for programmed death ligand 1 (PD-L1) and a chelating moiety, and optionally a linker, wherein the linker, when present connects the peptide and the chelating moiety, and when the linker is absent, the chelating moiety is attached directly to the peptide through a primary amine of an amino acid of the peptide, the method comprising:
(a) providing or preparing a peptide conjugate precursor solution comprising a conjugate of a peptide having a binding specificity for programmed death ligand 1 (PD-L1) and a chelating moiety, and optionally a linker;
(b) providing or preparing an anion exchange cartridge comprising [¹⁸F] ions;
(c) adding an aluminum chloride solution, acetonitrile, ascorbic acid, and the peptide precursor solution to a reaction vessel;
(d) eluting the anion exchange cartridge to release the [¹⁸F] ions into the reaction vessel;
(e) heating the reaction vessel for a period of time;
(f) cooling the reaction vessel;
(g) adding ascorbic acid to the cooled reaction vessel to form a solution comprising the ¹⁸F-labeled imaging agent;
(h) eluting the solution comprising the ¹⁸F-labeled imaging agent through a solid phase extraction cartridge;
(i) washing the solid phase extraction cartridge with ascorbic acid;
(j) eluting the solid phase extraction cartridge with an alkyl alcohol followed by sodium chloride for injection, USP with (+)-sodium L-ascorbate, through a sterilizing filter; and
(k) collecting the ¹⁸F-labeled imaging agent.

2. The method of claim 1, wherein the peptide having a binding specificity for PD-L1:
(a) interacts with amino acids Y56, E58, A113, M115, and Y123 of PD-L1;
(b) has at least 80% sequence identity to the peptide WL12, DK221, or DK222;
(c) has at least 85% sequence identity to the peptide WL12, DK221, or DK222;
(d) has at least 90% sequence identity to the peptide WL12, DK221, or DK222; and/or
(e) has 100% sequence identity to the peptide WL12, DK221, or DK222.

3-6. (canceled)

7. The method of claim 1, wherein the imaging agent comprises a compound of formula (I): 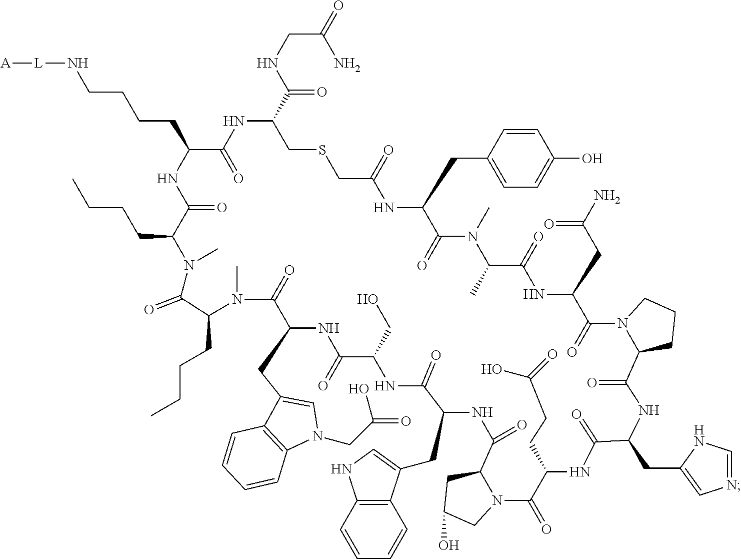
wherein:
L is a linker, which can be present or absent, and when present has the following general formula: 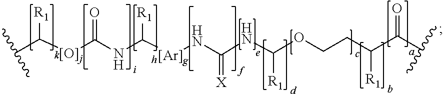
wherein:
X is S or O;
a, e, f, g, i, and j are each independently an integer selected the group consisting of 0 and 1;
b, d, h, and k are each independently an integer selected from the group consisting of 0, 1, 2, 3, 4, 5, 6, 7, and 8;
c is an integer having a range from 0 to 40;
each R₁ is H or —COOR₂, wherein R₂ is H or C₁-C₄ alkyl; Ar is substituted or unsubstituted aryl or heteroaryl; and
A is a chelating moiety.

8. The method of claim 7, wherein the linker is selected from the group consisting of: 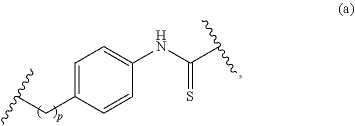
wherein p is an integer selected from 0, 1, 2, 3, and 4; 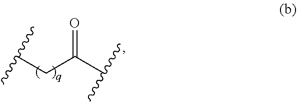
wherein q is an integer selected from the group consisting of 0, 1, 2, 3, 4, 5, 6, 7, and 8; 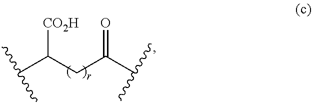
wherein r is an integer selected from the group consisting of 0, 1, 2, 3, 4, 5,6, 7, and 8; 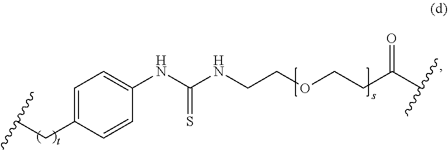
wherein s is an integer having a range from 1 to 40 and t is an integer selected from 0 or 1; 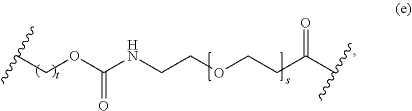
wherein s is an integer having a range from 1 to 40 and t is an integer selected from 0 or 1; and 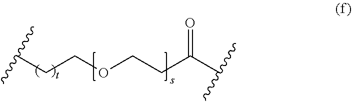
wherein s is an integer having a range from 1 to 40 and t is an integer selected from 0 or 1.

9. The method of claim 1, wherein the chelating moiety is selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTA-tris(t-butyl)ester, DOTAGA-(t-butyl)₄, DOTA-di(t-butyl)ester, DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), DO3A-(t-butyl), DO3AM (2,2′,2″-(1,4,7,10-tetraazacyclododecane-1,4,7-triyl)triacetamide), p-NH₂-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NOTA (t-butyl)₂, NO2A (1,4,7-Triazacyclononane-1,4-bis(acetic acid)-7-(acetamide), NODA (1,4,7-triazacyclononane-1,4-diacetate); NODAGA (1,4,7-triazacyclononane, 1-glutaric acid-4,7-acetic acid), NODAGA(t-butyl)₃, NOTAGA (1,4,7-triazonane-1,4-diyl)diacetic acid), DFO (Desferoxamine), DTPA (2-[Bis[2-[bis(carboxymethyl)amino]ethyl]amino]acetic acid), DTPA-tetra(t-butyl)ester (diethylenetriamine-N,N,N″,N″-tetra-tert-butyl acetate-N′-acetic acid), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3,6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), BaBaSar, tris(hydroxypyridinone) (THP), THP(benzyl)₃, NOPO (3-(((4,7-bis((hydroxy(hydroxymethyl)phosphoryl)-methyl)-1,4,7-triazonan-1-yl)methyl)(hydroxy)phosphoryl)propanoic acid), TRAP (3,3′,3″-(((1,4,7-triazonane-1,4,7-triyl)tris(methylene))tris(hydroxyphosphoryl))-tripropanoic acid), p-NH₂-Bn-PCTA (3,6,9,15-Tetraazabicyclo[9.3.1] pentadeca-1(15),11,13-triene-4-S-(4-aminobenzyl)-3,6,9-triacetic acid), biotin (5-[(3aS,4S,6aR)-2-oxohexahydro-1H-thieno[3,4-d]imidazol-4-yl]pentanoic acid).

10. The method of claim 9, wherein the chelating moiety is selected from the group consisting of:

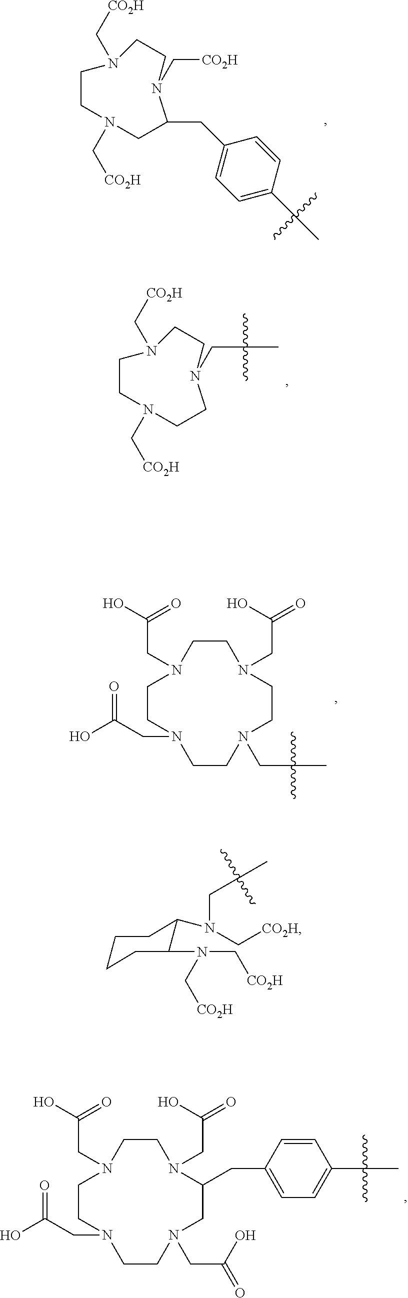 [SMILES: *c1ccc(CC2CN(CC(=O)O)CCN(CC(=O)O)CCN2CC(=O)O)cc1 ; *CN1CCN(CC(=O)O)CCN(CC(=O)O)CC1 ; *CN1CCN(CC(=O)O)CCN(CC(=O)O)CCN(CC(=O)O)CC1 ; *CN(CC(=O)O)C1CCCCC1N(CC(=O)O)CC(=O)O ; *c1ccc(CC2CN(CC(=O)O)CCN(CC(=O)O)CCN(CC(=O)O)CCN2CC(=O)O)cc1]

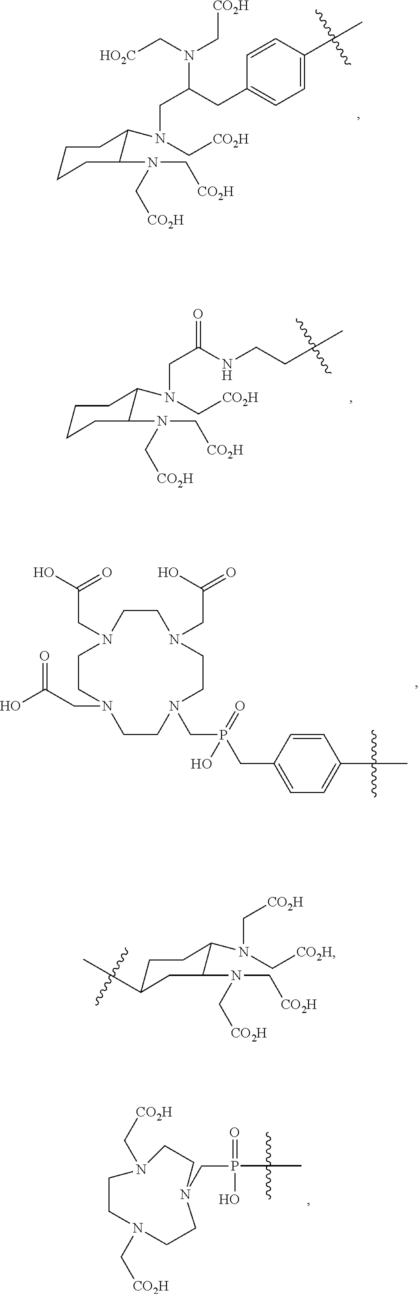 [SMILES: *c1ccc(CC(CN(CC(=O)O)C2CCCCC2N(CC(=O)O)CC(=O)O)N(CC(=O)O)CC(=O)O)cc1 ; *CCNC(=O)CN(CC(=O)O)C1CCCCC1N(CC(=O)O)CC(=O)O ; *c1ccc(CP(=O)(O)CN2CCN(CC(=O)O)CCN(CC(=O)O)CCN(CC(=O)O)CC2)cc1 ; *C1CCC(N(CC(=O)O)CC(=O)O)C(N(CC(=O)O)CC(=O)O)C1 ; *P(=O)(O)CN1CCN(CC(=O)O)CCN(CC(=O)O)CC1]

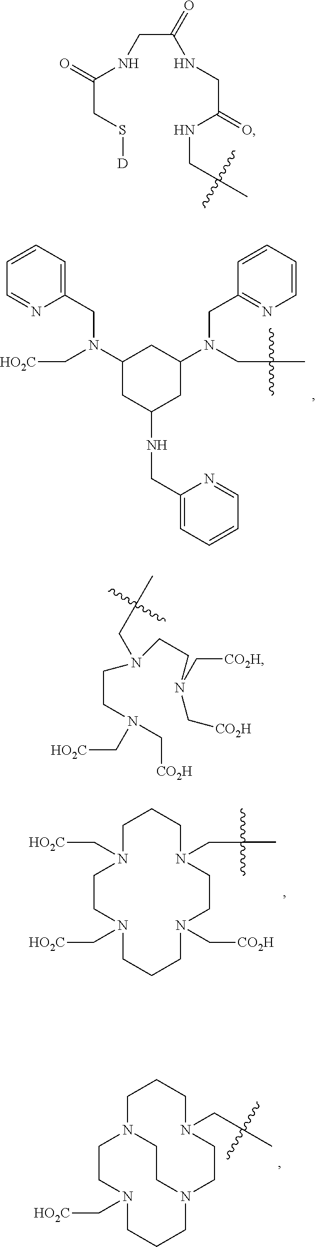

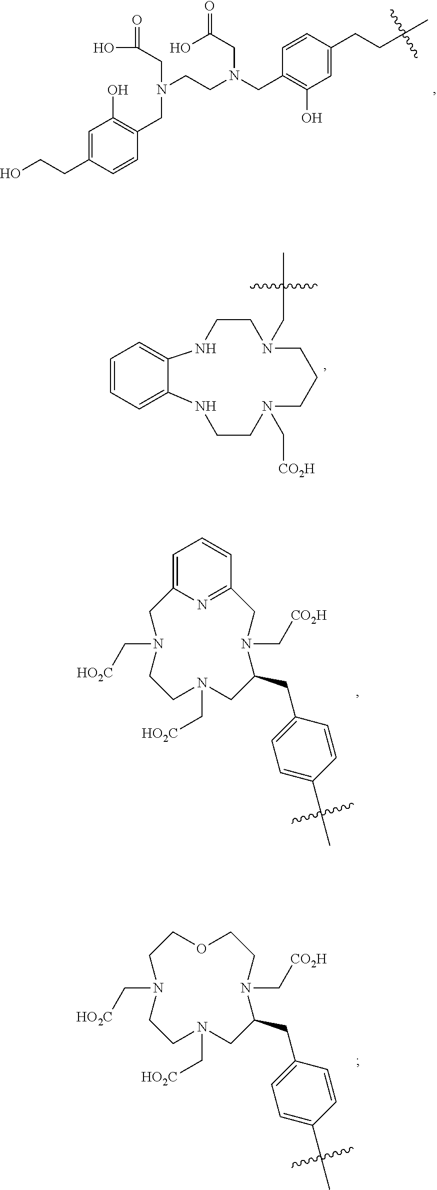 [SMILES: *CCc1ccc(CN(CCN(CC(=O)O)Cc2ccc(CCO)cc2O)CC(=O)O)c(O)c1 ; *CN1CCCN(CC(=O)O)CCNc2ccccc2NCC1 ; *c1ccc(C[C@H]2CN(CC(=O)O)CCN(CC(=O)O)Cc3cccc(n3)CN2CC(=O)O)cc1 ; *c1ccc(C[C@H]2CN(CC(=O)O)CCN(CC(=O)O)CCOCCN2CC(=O)O)cc1]

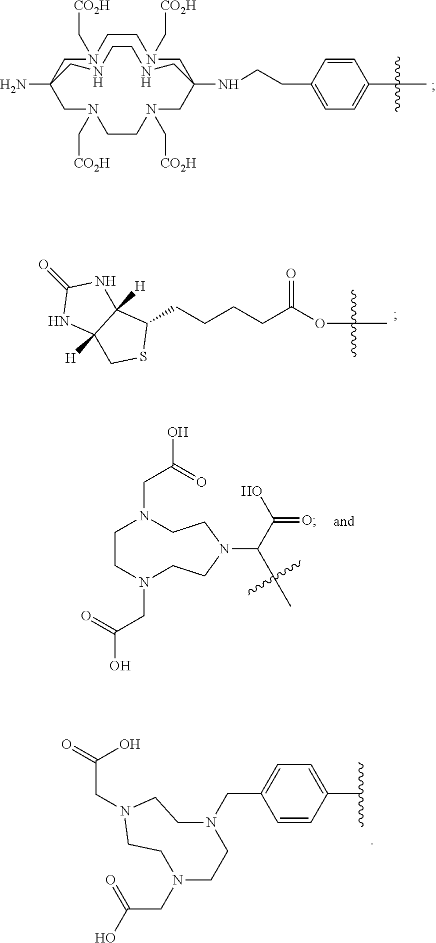 [SMILES: *c1ccc(CCNC23CNCCNCC(N)(CN(CC(=O)O)CCN(CC(=O)O)C2)CN(CC(=O)O)CCN(CC(=O)O)C3)cc1 ; *OC(=O)CCCC[C@@H]1SC[C@@H]2NC(=O)N[C@@H]21 ; *C(C(=O)O)N1CCN(CC(=O)O)CCN(CC(=O)O)CC1 ; *c1ccc(CN2CCN(CC(=O)O)CCN(CC(=O)O)CC2)cc1]

11. (canceled)

12. The method of claim 1, wherein the imaging agent is selected from the group consisting of: 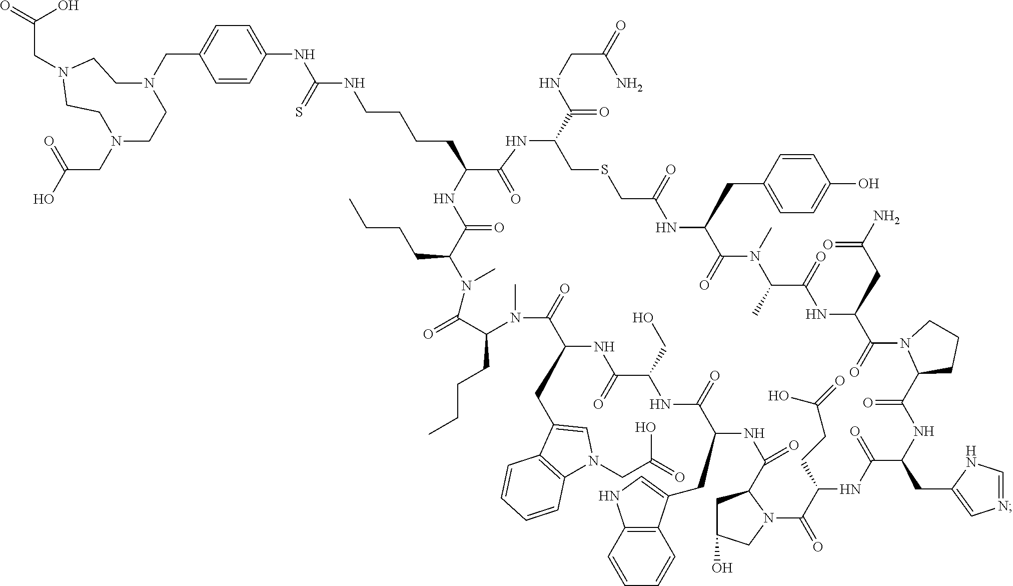 [SMILES: CCCC[C@H]1C(=O)N(C)[C@@H](CCCC)C(=O)N[C@@H](CCCCNC(=S)Nc2ccc(CN3CCN(CC(=O)O)CCN(CC(=O)O)CC3)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C] 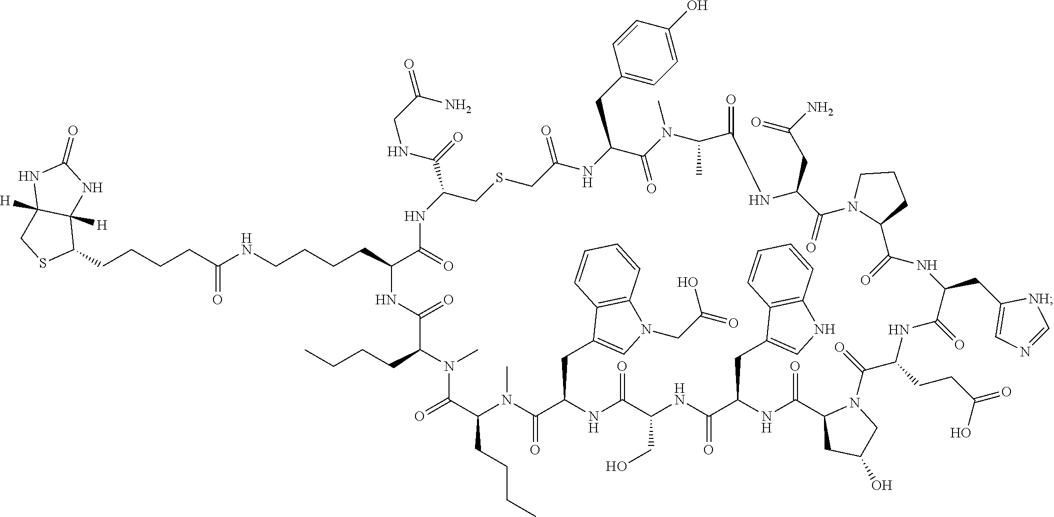 [SMILES: CCCC[C@H]1C(=O)N(C)[C@@H](CCCC)C(=O)N[C@@H](CCCCNC(=O)CCCC[C@@H]2SC[C@@H]3NC(=O)N[C@@H]32)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@H](CCC(=O)O)C(=O)N2C[C@H](O)C[C@H]2C(=O)N[C@H](Cc2c[nH]c3ccccc23)C(=O)N[C@H](CO)C(=O)N[C@H](Cc2cn(CC(=O)O)c3ccccc23)C(=O)N1C] 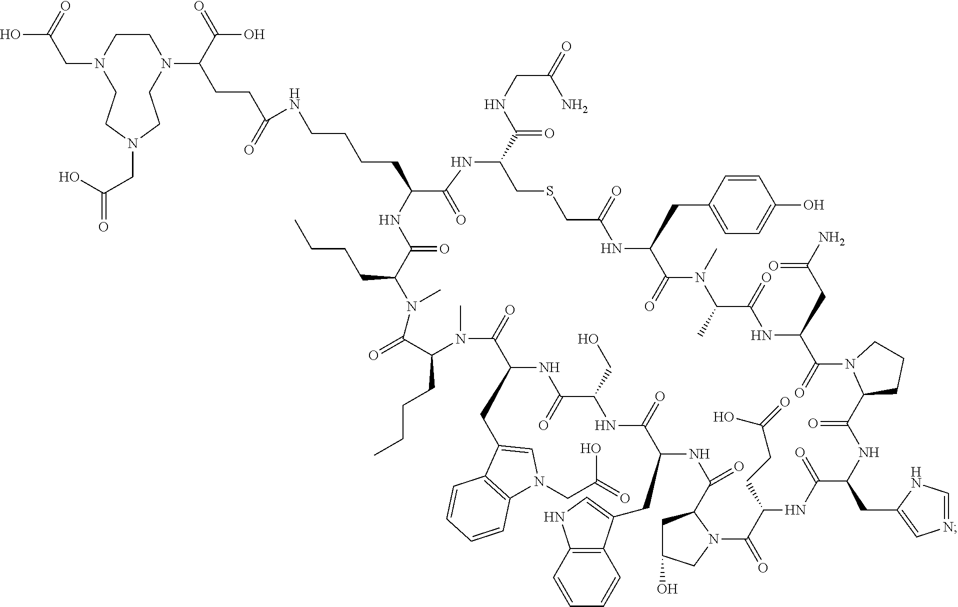 [SMILES: CCCC[C@H]1C(=O)N(C)[C@@H](CCCC)C(=O)N[C@@H](CCCCNC(=O)CCC(C(=O)O)N2CCN(CC(=O)O)CCN(CC(=O)O)CC2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C] 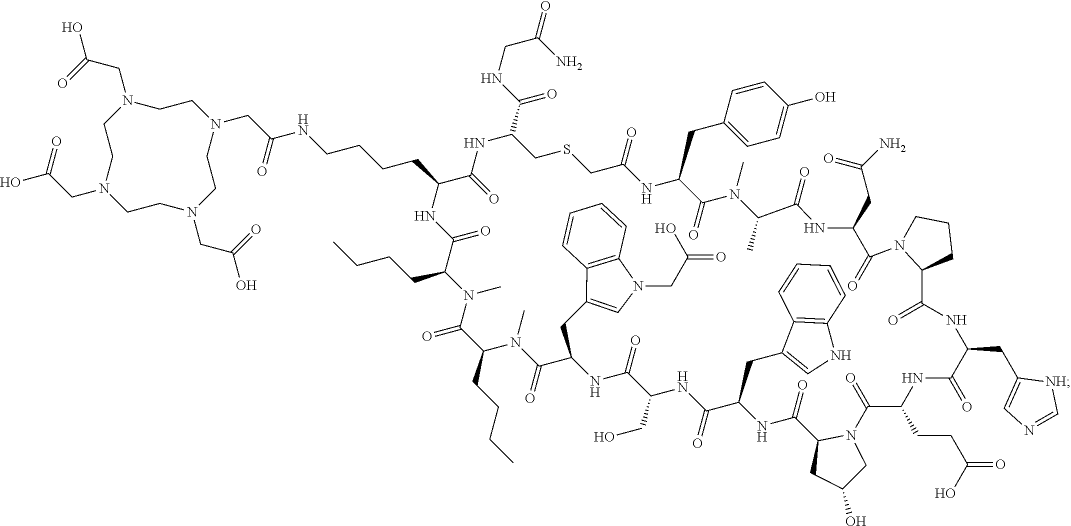 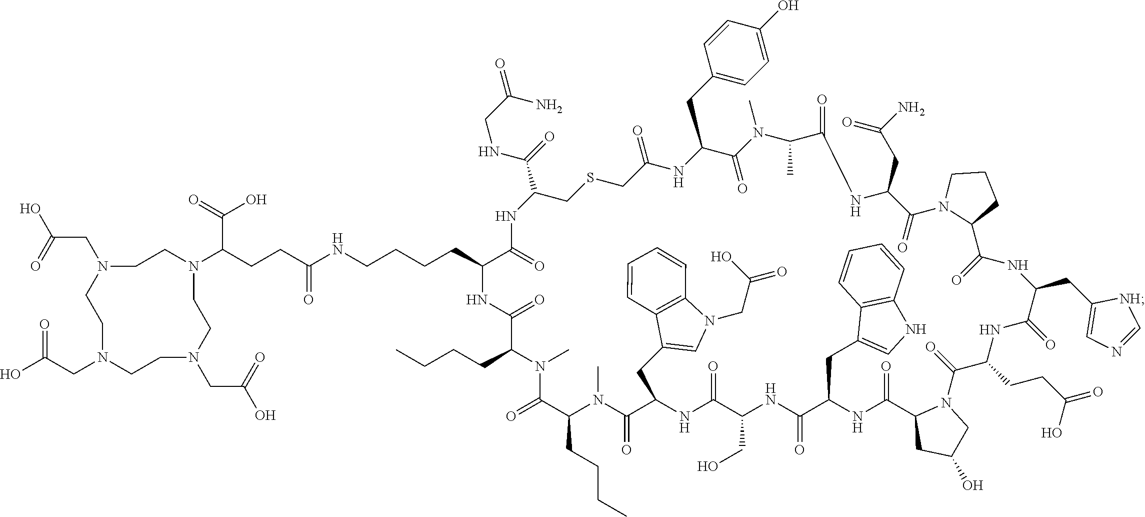 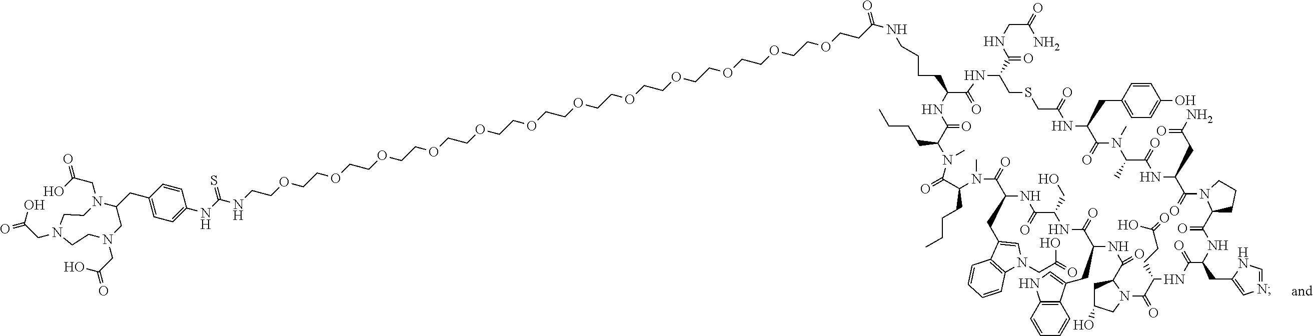 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCNC(=S)Nc2ccc(CC3CN(CC(=O)O)CCN(CC(=O)O)CCN3CC(=O)O)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C] 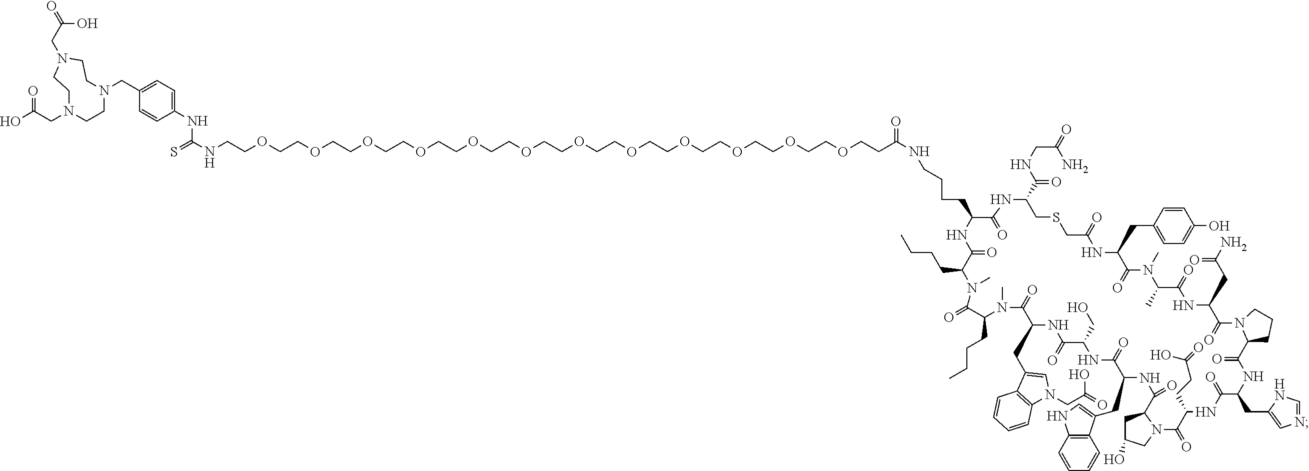 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCNC(=S)Nc2ccc(CN3CCN(CC(=O)O)CCN(CC(=O)O)CC3)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]
wherein the chelating moiety of each compound comprises Al[¹⁸F].

13. The method of claim 1, wherein the ¹⁸F-labeled imaging agent is [¹⁸F]DK222 having the following chemical structure:

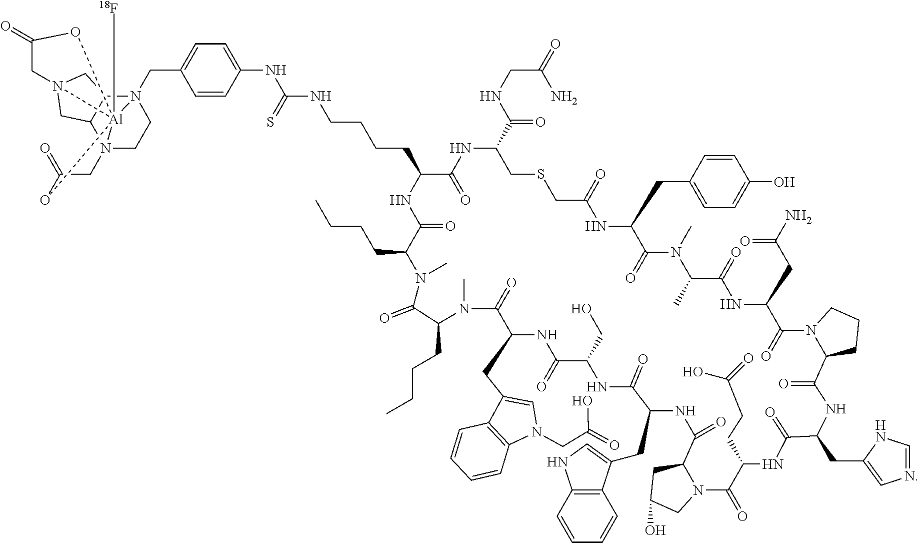

14. The method of claim 1, wherein:
(a) the aluminum chloride solution comprises an aluminum chloride hexahydrate solution in ascorbic acid;
(b) ascorbic acid at each occurrence comprises a 5-M ascorbic acid aqueous solution; and/or
(c) the anion exchange cartridge is eluted with a base.

15-16. (canceled)

17. The method of claim 14, wherein the base is potassium acetate.

18. The method of claim 1, wherein:
(a) the reaction vessel is heated to about 110° C.±20° C.;
(b) the reaction vessel is heated for about 10 minutes; and/or
(c) the reactive vessel is cooled to less than about 50° C.

19-20. (canceled)

21. The method of claim 1, wherein the solid phase extraction cartridge comprises a C18 or tC18 sorbent.

22. The method of claim 1, wherein the alkyl alcohol is selected from the group consisting of methanol, ethanol, propyl alcohol, isopropyl alcohol, and butanol.

23. (canceled)

24. The method of claim 1, wherein the sterilizing filter comprises a polytetrafluoroethylene membrane.

25. The method of claim 1, wherein one or more steps (a)-(k) are automated.

26. The method of claim 1, wherein the ¹⁸F-labeled imaging agent meets one or more quality control criteria selected from the group consisting of:
(a) a clear, colorless solution with no visible particulate matter;
(b) a yield not less than 20 mCi of the ¹⁸F-labeled imaging agent;
(c) a pH in the range of 3.5 to 5.5;
(d) the sterilizing filter has a greater than or equal to 13 psi bubble-point test;
(e) a radionuclidic identity and purity in the range of 105-115 minutes for half-life measurement;
(f) a radiochemical purity of 90% or greater for the ¹⁸F-labeled imaging agent;
(g) a specific activity greater than 250 mCi/□mole;
(h) less than 10% ethanol and less than 273 parts per million acetonitrile residual solvent; and
(i) bacterial endotoxins equal to or less than eleven USP endotoxin units per mL.

27. The method of claim 26, further comprising inoculating the ¹⁸F-labeled imaging agent at the end of each production day and monitoring for 14 days for visible growth of microorganisms.

28. The method of claim 1, wherein:
(a) the [¹⁸F] ions comprising the anion exchange cartridge having a range from about 100 mCi to greater than about 1,000 mCi;
(b) the ¹⁸F-labeled imaging agent has a radioactivity having a range from about 50 mCi to greater than about 500 mCi;
(c) the ¹⁸F-labeled imaging agent has a yield at the end of synthesis (EOS) having a range from about 30% to about 60%;
(d) the ¹⁸F-labeled imaging agent has a specific activity having a range from about 2,000 mCi/μmole to greater than about 25,000 mCi/μmole;
(e) the ¹⁸F-labeled imaging agent has an effective specific activity having a range from about 900 mCi/μmole to about 8,000 mCi/μmole; and/or
(f) the ¹⁸F-labeled imaging agent has a radiochemical activity having a range from about 90% to about 100%.

29-33. (canceled)

34. A kit for preparing an ¹⁸F-labeled imaging agent for detecting Programmed Death Ligand 1 (PD-L1), the kit comprising a conjugate of a peptide having a binding specificity for programmed death ligand 1 (PD-L1) and a chelating moiety, and optionally a linker, and optionally one or more of an anion exchange cartridge comprising [¹⁸F] ions, a solid phase extraction cartridge, and one or more solvents or reagents.

35-37. (canceled)
